(19) United States
(12) Patent Application Publication
(10) Pub. No.: US 2025/0169721 A1
(43) Pub. Date: May 29, 2025

(54) GLUCOSE MONITORING ALARM DEVICE AND RELATED FUNCTIONALITY

(71) Applicant: Custom TypeOne LLC, Stow, OH (US)

(72) Inventors: Chris Fawcett, Charlotte, NC (US); Jonathan Eric Fawcett, Stow, OH (US)

(21) Appl. No.: 18/943,187

(22) Filed: Nov. 11, 2024

Related U.S. Application Data
(60) Provisional application No. 63/602,789, filed on Nov. 27, 2023.

(51) Int. Cl. A61B 5/145; A61B 5/00

(52) U.S. Cl. CPC A61B 5/14532; A61B 5/4839; A61B 5/7282; A61B 5/742; A61B 5/746

(57) ABSTRACT

A notification device is provided for presenting blood glucose level information for a tracked individual. The notification device is configured to receive first and second blood glucose level information from first and second tracking devices associated with different manufacturers. The first and second blood glucose level information each include at least one of a blood glucose level or a first or second derivative for the blood glucose level as a function of time. The notification device comprises a screen, processor(s), and memory device(s) containing computer readable code. When executed by the processor(s), the computer readable code is configured to cause the processor(s) to receive the first and/or the second blood glucose level information and cause presentation of the first and/or the second blood glucose level information on the screen. The first and second tracking devices are each a continuous blood glucose monitor.

### CROSS-REFERENCE TO RELATED APPLICATIONS

This application claims priority to U.S. Provisional Application No. 63/602,789, filed Nov. 27, 2023, entitled “Glucose Monitoring Alarm Device and Related Functionality,” which is incorporated herein by reference in its entirety.

### FIELD

Embodiments relate generally to notification devices for monitoring blood glucose levels for individuals and generating alerts.

### BACKGROUND

Significant health issues arise when a person's blood glucose level is too high, with people potentially having issues with vomiting or potentially falling into a coma. Additionally, significant health issues may arise when a person's blood glucose level is too low, with such individuals potentially losing consciousness. Thus, many people with diabetes, hypoglycemia, or other diseases that affect blood glucose levels frequently monitor their blood glucose level to ensure that these levels are appropriate.

Continuous glucose monitors are often used by those with diabetes to measure blood glucose levels for tracked individuals, and data from these continuous glucose monitors is often sent to a smart phone of the user (e.g., the wearer, a guardian of the wearer, a healthcare provider of the wearer, etc.) and/or to remote server(s). However, users can often forget to regularly monitor the blood glucose levels when relevant data is merely available in a phone application, and a user may unknowingly continue with what they are doing without realizing that their blood glucose level is poor or very poor and, sometimes, in need of immediate attention. Further, when a user or a tracked individual is sleeping, such notifications may be critical. In cases of outages in cellular service, power outages, and in other similar instances, the phone and other servers may not be capable of obtaining data from continuous glucose monitors, preventing the user from being able to reliably track blood glucose levels. This issue may also be present where the phone is out of power, set on do-not-disturb, in a sleep mode, in an airplane mode, etc. Alerts are oftentimes presented in a standard fashion where the alert does not change, and this leads to brain habituation for the user. Additionally, when only audio alerts are generated, a maximum volume for a phone is often insufficient to wake some people from sleep.

Various devices and applications used for tracking blood glucose levels merely generate alerts based on the blood glucose levels alone and do not account for other factors that may be present. By not considering other factors, the devices and applications are less accurate in determining when alerts are necessary, generating more false alarms when no alert is necessary and also generating alerts in a delayed fashion in some instances.

### BRIEF SUMMARY

In various example embodiments provided herein, users are able to track blood glucose levels with a notification device. The notification device may present information to the user in a readily understandable manner, with the blood glucose level presented alongside other information. A trend arrow may also be presented that is representative of the rate of change or delta for the blood glucose level. Icons, symbols, emojis, quotes, slang, or other indicators may also be presented that are dependent on the blood glucose levels as well. For example, a unicorn image may be presented on the screen to indicate a “unicorn” blood glucose level, a range of Urgent Low, Low, In Range, High, and Urgent High may be provided to indicate the current blood glucose level for the tracked individuals, an alphanumeric grade may be provided to indicate the current blood glucose level for the tracked individuals, a color may be provided to indicate the current blood glucose level, etc. Information may be presented with emphasis in certain scenarios such as when the blood glucose levels are poor and need attention. Alerts may also be generated, with the alerts being generated by vibration of the notification device, a component therein, or a component attached to the device, by audible sounds generated by the notification device or a component therein, by flashing certain portions of the screen to provide a visual alert, etc. Alerts may be provided in a randomized manner to prevent brain habituation.

In some embodiments, the indicators may be animated such that the material presented on any screen changes over time, thereby providing the appearance that the material is moving. For example, an emoji or another image may be provided, and the emoji or image may move over time to provide the appearance of movement.

In some embodiments, the notification device may be configured to obtain data regarding blood glucose levels and/or other information from a number of different data providers (e.g., different manufacturers), allowing the device to be used to track blood glucose levels for multiple tracked individuals, with different data providers being used to track data for the different tracked individuals. In some embodiments, where a tracked individual is using a first tracking device associated with a first manufacturer that uses a first communication protocol and decides to switch to a second tracking device associated with a second manufacturer that uses a second communication protocol that is different from the first communication protocol, the user may still continue to use the notification device since the notification device may have connectivity to devices made by the first manufacturer, the second manufacturer, and other manufacturers or to data feeds for the different manufacturers.

In some embodiments, the notification device may be used to track blood glucose data for multiple tracked individuals. This may be beneficial where multiple people within a household have diabetes or otherwise need to track their blood glucose levels. Where the notification device is used to present blood glucose levels for multiple tracked individuals, the information associated with each tracked individual may be presented in a certain color to identify the tracked individual or other indications may be provided to indicate that the information presented is associated with a particular tracked individual.

In some embodiments, the notification device may (via processing in the device and/or remotely located device(s)/server(s)) determine whether or not to alarm based on a trend in the wearer's blood glucose level. In this regard, the notification device may take into account whether a wearer's blood glucose level may be adjusting to come back into normal range such that the user does not need to be notified via an alarm (or via certain levels of alarm functionality). For example, if the blood glucose level is going to correct itself and come back into a desirable range, it may not make sense to set off an alarm, such as an alarm that may awaken the user needlessly.

In some embodiments, a user (e.g., a tracked individual) may activate a button for some duration of time to generate an alert signal that may be sent to one or more other recipients. This may provide added safety benefits in instances where the tracked individual's blood glucose level is extremely high or extremely low, allowing the tracked individual to alert others to the situation so that the tracked individual may obtain medical assistance. Activation of the button may cause an alert to be generated at another notification device or another device such as a user's phone, tablet, etc.

In some embodiments, the notification device may function as a continuous glucose monitor secondary display, with the notification device not being configured to provide any alerts. In other embodiments, the notification device may function as a continuous glucose monitor secondary alarm system, with the notification device being configured to provide alerts. The notification device may be a secondary device that does not directly communicate with the actual on-body medical device, and the notification device may accomplish this by, for example, downloading data from the cloud or in other ways.

In an example embodiment, a notification device for presenting blood glucose level information for a tracked individual is provided. The notification device is configured to receive first blood glucose level information from a first tracking device associated with a first manufacturer, the notification device is configured to receive second blood glucose level information from a second tracking device associated with a second manufacturer, and the first blood glucose level information and the second blood glucose level information each include a blood glucose level, a first derivative for the blood glucose level as a function of time, and/or a second derivative for the blood glucose level as a function of time. The notification device comprises a screen, one or more processors, and one or more memory devices containing computer readable code thereon. When executed by the processor(s), the computer readable code is configured to cause the processor(s) to receive the first blood glucose level information and/or the second blood glucose level information and to cause presentation of the first blood glucose level information and/or the second blood glucose level information on the screen. The first tracking device and the second tracking device are each a continuous blood glucose monitor.

In some embodiments, the first blood glucose level information may be associated with blood glucose levels for a first tracked individual, and the second blood glucose level information may be associated with blood glucose levels for a second tracked individual.

In some embodiments, the notification device may be configured to generate alerts based on the first blood glucose level information, the alerts may include audio alerts, visual alerts, vibratory alerts, and/or notifications, and the notifications may be sent to another device and may include a phone call, a text message, an email, and/or a notification alert. Additionally, in some embodiments, the notification device may be configured to cause randomized alerts. Furthermore, in some embodiments, the notification device may be configured to enable customization of time periods when the alerts are enabled and when the alerts are disabled.

In some embodiments, the notification device may be configured to enable snoozing of alerts. In some embodiments, when executed by the processor(s), the computer readable code may be configured to cause the processor(s) to receive a snooze command from another device and to cause an alert to be snoozed based on the snooze command. In some embodiments, when executed by the processor(s), the computer readable code may be configured to cause the processor(s) to receive a snooze command and to cause an alert at the notification device and a second alert at a second device to be snoozed based on the snooze command. In some embodiments, when executed by the processor(s), the computer readable code may be configured to cause the processor(s) to generate a first alert at a first time and to generate a second alert at a second time after the first time, with the second alert being greater in magnitude relative to the first alert when the notification device has not been snoozed between the first time and the second time.

In some embodiments, when executed by the processor(s), the computer readable code may be configured to cause the processor(s) to receive an indication that the blood glucose level either falls inside or outside of an acceptable range and to cause an alert to be generated if the blood glucose level falls outside of the acceptable range, with the indication not including a specific blood glucose level. Furthermore, in some embodiments, when executed by the processor(s), the computer readable code may be configured to cause the processor(s) to receive a second indication including a specific blood glucose level, and the indication may be received at a greater frequency than the second indication.

In some embodiments, when executed by the processor(s), the computer readable code may be configured to cause the processor(s) to receive data from an insulin pump and to cause the data received from the insulin pump to be displayed. In some embodiments, the notification device may be configured to generate alerts indicating when limited data is available or when no data is available.

In another example embodiment, a method for presenting blood glucose level information for a tracked individual is provided. The method comprises receiving first blood glucose level information and/or second blood glucose level information, and the method also comprises causing presentation of the first blood glucose level information and/or the second blood glucose level information on a screen of a notification device. The first blood glucose level information is from a first tracking device associated with a first manufacturer, the second blood glucose level information is from a second tracking device associated with a second manufacturer, and the first blood glucose level information and the second blood glucose level information each include a blood glucose level, a first derivative for the blood glucose level as a function of time, and/or a second derivative for the blood glucose level as a function of time. Additionally, the first tracking device and the second tracking device are each a continuous blood glucose monitor.

In some embodiments, the first blood glucose level information may be associated with blood glucose levels for a first tracked individual, and the second blood glucose level information may be associated with blood glucose levels for a second tracked individual. In some embodiments, the method may also include receiving data from an insulin pump and causing the data received from the insulin pump to be displayed.

In some embodiments, the method may also include causing generation of alerts based on the first blood glucose level information. The alerts may include an audio alert, a visual alert, a vibratory alert, and/or a notification, and the notification may be sent to another device and may include a phone call, a text message, an email, and/or a notification alert. Furthermore, in some embodiments, the alerts may be randomized relative to each other. Additionally, in some embodiments, the method may also include receiving a customized schedule from a user with time periods when alerts are enabled and disabled, and the generation of the alerts may occur in a time period when alerts are enabled in the customized schedule. In some embodiments, the method may also include receiving a snooze command and causing the alerts to be snoozed based on the snooze command. Furthermore, in some embodiments, the method may also include generating a first alert at a first time and generating a second alert at a second time after the first time when the notification device has not been snoozed between the first time and the second time, with the second alert being greater in magnitude relative to the first alert. In some embodiments, the method may also include receiving a snooze command from another device and causing an alert to be snoozed based on the snooze command. In some embodiments, the method may also include receiving a snooze command and causing a first alert at the notification device and a second alert at a second device to be snoozed based on the snooze command.

In some embodiments, the method may also include receiving an indication that the blood glucose level either falls inside or outside of an acceptable range, with the indication not including a specific blood glucose level, and the method may also include causing an alert to be generated if the blood glucose level falls outside of the acceptable range. Furthermore, in some embodiments, the method may also include receiving a second indication including a specific blood glucose level, and the indication may be received at a greater frequency than the second indication. In some embodiments, the method may also include generating alerts indicating when limited data is available or when no data is available.

In another example embodiment, a notification device for presenting blood glucose level information for a tracked individual is provided. The notification device includes a screen, one or more processors, and one or more memory devices containing computer readable code thereon that. When executed by the processor(s), the computer readable code is configured to cause the processor(s) to perform various actions. These actions include receiving first blood glucose level information from a first tracking device worn by the tracked individual. The first blood glucose level information includes a blood glucose level for the tracked individual, a first derivative for the blood glucose level of the tracked individual as a function of time, and/or a second derivative for the blood glucose level of the tracked individual as a function of time. The actions also include determining, based on the first blood glucose level information, a representation associated with the first blood glucose level information, and the representation includes an emoji, a slang term, a color, and/or a quote. The actions also include causing presentation of the first blood glucose level information and the representation on the screen. The first tracking device is a continuous blood glucose monitor.

In some embodiments, the representation may be customizable by the tracked individual or by another person so that customized representations are presented for different blood glucose level ranges.

In some embodiments, the notification device may be configured to generate alerts based on the first blood glucose level information, the alerts may comprise an audio alert, a visual alert, a vibratory alert, and/or a notification, and the notification may be sent to another device and may include a phone call, a text message, an email, and/or a notification alert. Furthermore, in some embodiments, the notification device may be configured to cause randomized alerts. Also, in some embodiments, the notification device may be configured to enable snoozing of alerts by a user. In some embodiments, when executed by the processor(s), the computer readable code may be configured to cause the processor(s) to receive a snooze command from another device and to cause an alert to be snoozed based on the snooze command. Additionally, in some embodiments, when executed by the processor(s), the computer readable code may be configured to cause the processor(s) to receive a snooze command, to cause an alert at the notification device to be snoozed based on the snooze command, and to cause a second alert at a second device to be snoozed based on the snooze command.

In some embodiments, when executed by the processor(s), the computer readable code may be configured to cause the processor(s) to generate a first alert at a first time and to generate a second alert at a second time after the first time. The second alert may be greater in magnitude relative to the first alert when the notification device has not been snoozed between the first time and the second time.

In some embodiments, the notification device may be configured to enable customization of time periods when the alerts are enabled and when alerts are disabled.

In some embodiments, the notification device may include an alert button. When executed by the processor(s), the computer readable code may be configured to cause the processor(s) to receive an alert command upon the alert button being pressed by a user and to cause an alert to be generated based on the alert command.

In some embodiments, when executed by the processor(s), the computer readable code may be configured to cause the processor(s) to receive data from an insulin pump and to cause the data received from the insulin pump to be displayed.

In some embodiments, when executed by the processor(s), the computer readable code may be configured to cause the processor(s) to receive the second blood glucose level information from a second tracking device worn by a second tracked individual and to cause presentation of the second blood glucose level information on the screen alongside the first blood glucose level information. The second blood glucose level information may include a blood glucose level for the tracked individual as a function of time, a first derivative of the blood glucose level for the second tracked individual as a function of time, and/or a second derivative of the blood glucose level for the second tracked individual as a function of time.

In some embodiments, when executed by the processor(s), the computer readable code may be configured to cause the processor(s) to receive an indication that the blood glucose level either falls inside or outside of an acceptable range, with indication not including a specific blood glucose level. In some embodiments, when executed by the processor(s), the computer readable code may be configured to cause the processor(s) to receive a second indication including a specific blood glucose level, and the indication may be received at a greater frequency than the second indication. In some embodiments, some embodiments, the notification device may be configured to generate alerts indicating when limited data is available or when no data is available.

In another example embodiment, a method for presenting blood glucose level information for a tracked individual is provided. The method comprises receiving first blood glucose level information from a first tracking device worn by the tracked individual. The first blood glucose level information includes a blood glucose level for the tracked individual, a first derivative for the blood glucose level of the tracked individual as a function of time, and/or a second derivative for the blood glucose level of the tracked individual as a function of time. The method also includes determining, based on the first blood glucose level information, a representation associated with the first blood glucose level information. The representation includes an emoji, a slang term, a color, and/or a quote. The method also includes causing presentation of the first blood glucose level information and the representation on the screen. The first tracking device is a continuous blood glucose monitor.

In some embodiments, the representation may be customizable by the tracked individual or by another person so that customized representations are presented for different blood glucose level ranges.

In some embodiments, the method may also include causing an alert to be generated based on the first blood glucose level information. The alert may comprise an audio alert, a visual alert, a vibratory alert, and/or a notification, and the notification may be sent to another device and includes a phone call, a text message, an email, and/or a notification alert. Additionally, in some embodiments, alerts may be randomized. Furthermore, in some embodiments, the method may also include receiving a customized schedule from a user with time periods when alerts are enabled and disabled, and generation of the alerts may occur in a time period when alerts are enabled in the customized schedule. Also, in some embodiments, the method may also include receiving an indication that the blood glucose level either falls inside or outside of an acceptable range with the indication not including a specific blood glucose level, the method may also include receiving a second indication comprising a specific blood glucose level, and the indication may be received at a greater frequency than the second indication. In some embodiments, the alert may be based on the indication. Furthermore, in some embodiments, the method may also include receiving a snooze command and causing the alert to be snoozed based on the snooze command. Also, in some embodiments, a device where the snooze command is received may be different from a device where the alert is being generated. In some embodiments, the method may also include causing a second alert at a second device to be snoozed based on the snooze command. Furthermore, in some embodiments, the method may also include generating the alert at a first time and generating a second alert at a second time after the first time, with the second alert being greater in magnitude relative to the alert when the notification device has not been snoozed between the first time and the second time.

In some embodiments, the method may also include receiving an alert command and causing an alert to be generated at another device based on the alert command. In some embodiments, the method may also include receiving data from an insulin pump and causing the data received from the insulin pump to be displayed. In some embodiments, the method may also include receiving the second blood glucose level information from a second tracking device worn by a second tracked individual and causing presentation of the second blood glucose level information on the screen alongside the first blood glucose level information. The second blood glucose level information may include a blood glucose level for the tracked individual as a function of time, a first derivative of the blood glucose level for the second tracked individual as a function of time, and/or a second derivative of the blood glucose level for the second tracked individual as a function of time. In some embodiments, the method may also include generating alerts indicating when limited data is available or when no data is available.

In another example embodiment, a notification device for presenting blood glucose level information for a tracked individual is provided. The notification device comprises one or more processors and one or more memory devices containing computer readable code. When executed by the processor(s), the computer readable code is configured to cause the processor(s) to perform various actions. These actions include receiving the blood glucose level information from a first tracking device, and the blood glucose level information includes a blood glucose level. The actions also include determining a first derivative for the blood glucose level as a function of time and/or a second derivative for the blood glucose level as a function of time, determining whether an alert should be generated based on the blood glucose level and the first derivative and/or the second derivative for the blood glucose level as a function of time, causing the alert to be generated upon a determination that the alert should be generated, and refraining from causing the alert to be generated upon a determination that the alert should not be generated. Additionally, the first tracking device is a continuous blood glucose monitor.

In some embodiments, the determination may be made that the alert should be generated when the blood glucose level is in a normal range but when the first derivative for the blood glucose level indicates that the blood glucose level is about to exit the normal range. In some embodiments, the determination may be that the alert should not be generated, and the blood glucose level may fall outside of a normal range but a first derivative for the blood glucose level may indicate that the blood glucose level is about to enter into the normal range.

In another example embodiment, a method for presenting blood glucose level information for a tracked individual is provided. The method includes receiving the blood glucose level information from a first tracking device, with the blood glucose level information including a blood glucose level. The method also includes determining a first derivative for the blood glucose level as a function of time and/or a second derivative for the blood glucose level as a function of time, determining whether an alert should be generated based on the blood glucose level and the first derivative and/or the second derivative for the blood glucose level as a function of time, and causing the alert to be generated upon a determination that the alert should be generated.

In some embodiments, the determination of whether an alert should be generated may be made when the blood glucose level is in a normal range but the first derivative for the blood glucose level indicates that the blood glucose level is about to exit the normal range. Additionally, in some embodiments, the determination of whether an alert should be generated may result in a determination that the alert should not be generated, and the blood glucose level may fall outside of a normal range but a first derivative for the blood glucose level may indicate that the blood glucose level is about to enter into the normal range.

In another example embodiment, a notification device for presenting blood glucose level information for a tracked individual is provided. The notification device may be configured to receive first blood glucose level information from a first tracking device, and the first blood glucose level information may include a blood glucose level, a first derivative for the blood glucose level as a function of time, and/or a second derivative for the blood glucose level as a function of time. The notification device may include a button, one or more processors, and one or more memory devices containing computer readable code thereon. When executed by the processor(s), the computer readable code may be configured to cause the processor(s) to receive an indication that the button has been activated or that a voice command has been received and to cause an alert signal to be sent to a device upon receiving the indication. Additionally, the first tracking device may be a continuous blood glucose monitor. In some embodiments, the indication may be an indication that the button has been activated for a threshold period of time.

### BRIEF DESCRIPTION OF THE DRAWINGS

Reference will now be made to the accompanying drawings, which are not necessarily drawn to scale, and wherein:

FIG. 1 is a front, perspective view illustrating an example notification device, in accordance with some embodiments discussed herein;

FIG. 2 is a rear, perspective view illustrating an example notification device, in accordance with some embodiments discussed herein;

FIG. 3 is a front view illustrating an example notification device, in accordance with some embodiments discussed herein;

FIG. 4 is a top view illustrating an example notification device, in accordance with some embodiments discussed herein;

FIG. 5 is a right side view illustrating an example notification device, in accordance with some embodiments discussed herein;

FIG. 6 is a left side view illustrating an example notification device, in accordance with some embodiments discussed herein;

FIG. 7 is a front, perspective view illustrating an example notification device that shows an emoji representation indicative of a tracked individual's current blood glucose level, in accordance with some embodiments discussed herein;

FIG. 8 is a front, perspective view illustrating an example notification device that shows an enlarged blood glucose level and a trend arrow, in accordance with some embodiments discussed herein;

FIG. 9 is a front, perspective view illustrating an example notification device that is configured to present information for two different tracked individuals, in accordance with some embodiments discussed herein;

FIG. 10 is a front, perspective view illustrating an example notification device that includes the blood glucose level, the change in that level over a time interval, and a trend arrow, in accordance with some embodiments discussed herein;

FIG. 11 illustrates a continuous glucose monitor attached on an arm of a tracked individual, in accordance with some embodiments discussed herein;

FIG. 12 is a block diagram illustrating various components of a system for operating a notification device, in accordance with some embodiments discussed herein;

FIG. 13 illustrates graphs showing blood glucose levels as a function of time with blood glucose levels approaching a lower threshold of a normal or desired zone for blood glucose levels, in accordance with some embodiments discussed herein;

FIG. 14 illustrates an example insulin pump and a smart insulin pen, in accordance with some embodiments discussed herein;

FIG. 15 illustrates an example flow chart with a method for presenting blood glucose level information for a tracked individual, in accordance with some embodiments discussed herein;

FIG. 16 illustrates an example flow chart with a method for presenting blood glucose level information for a tracked individual, in accordance with some embodiments discussed herein; and

FIG. 17 illustrates an example flow chart with a method for presenting blood glucose level information for a tracked individual, in accordance with some embodiments discussed herein.

### DETAILED DESCRIPTION

Example embodiments now will be described more fully hereinafter with reference to the accompanying drawings, in which some, but not all embodiments are shown. Like reference numerals generally refer to like elements throughout. For example, reference numerals 100, 200, 300, etc. are each associated with a notification device. Additionally, connections or attachments may be direct or indirect connections or attachments unless specifically noted otherwise. As used herein, a “tracked individual” is a patient or some person whose blood glucose level or other data is being tracked. Furthermore, a “user” is any person that may be using the notification devices (or other device) described herein, including but not limited to tracked individual, a caregiver, a loved one of that person, a friend of that person, a coworker of that person, etc. Also, as used herein, a “tracking device” may be a glucose monitor such as a continuous blood glucose monitor, an insulin pump, or a smart insulin pen.

Example notification devices described herein are provided herein that may beneficially allow users to track blood glucose levels or other health information related to one or more tracked individuals. Notification devices may read blood glucose levels, such as from a phone of one or more users, and these blood glucose levels may be received over a wireless connection such as Bluetooth. Where Bluetooth technology is utilized to obtain blood glucose levels, the blood glucose levels may be transferred effectively even when no Wi-Fi or internet is available.

Notification devices may be configured to connect to different data providers. For example, notification devices may connect to devices provided by Dexcom®, Libre®, Medtronic®, Senseonics®, and other data providers. In order to do so, the notification devices may be configured to connect to various devices using different communication protocols. For example, different manufacturers or tracking devices may provide such data for usage in a format specific to the manufacturer and/or that may be accessible in a unique or otherwise manufacturer specific communication protocol. Example notification devices are configured to utilize multiple different manufacturer specific communication protocol(s) and/or receive multiple different types of format specific data—and still achieve the desired functionality described herein. This, of course, may be enabled by processing that may be adaptable to the multiple different manufacturers so that the purchaser and user may be confident that the notification device will operate as intended regardless of which tracking device is being used. This may enable notification devices to track data from a first data provider for a first tracked individual and to track data from a second data provider for a second tracked individual. This may also enable notification devices to track data from a first data provider for a tracked individual and then also allow the tracked individual to track data from a second data provider if the tracked individual begins to use a different blood glucose monitor or another tracking device. Notification devices may obtain blood glucose data or other data from the cloud in some embodiments so that notification devices may present the appropriate information thereon. However, notification devices may receive blood glucose data or other data via other communication approaches such as through a wired connection, through a wireless communication technology such as Bluetooth, Bluetooth Low Energy (BLE), Wi-Fi, or some other technology.

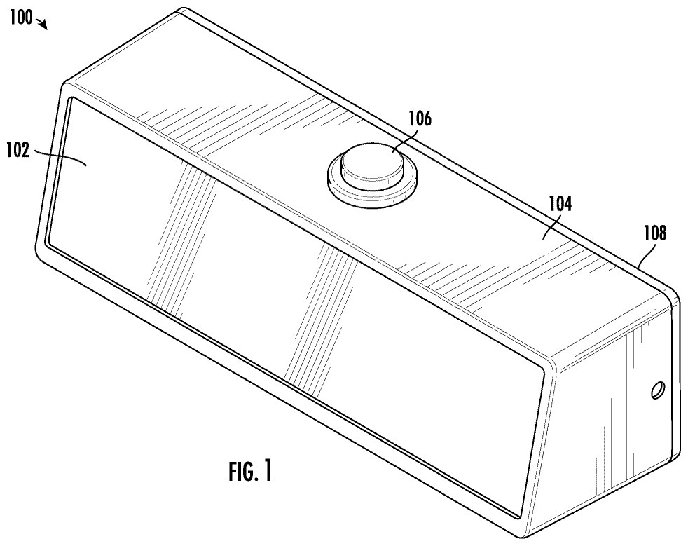

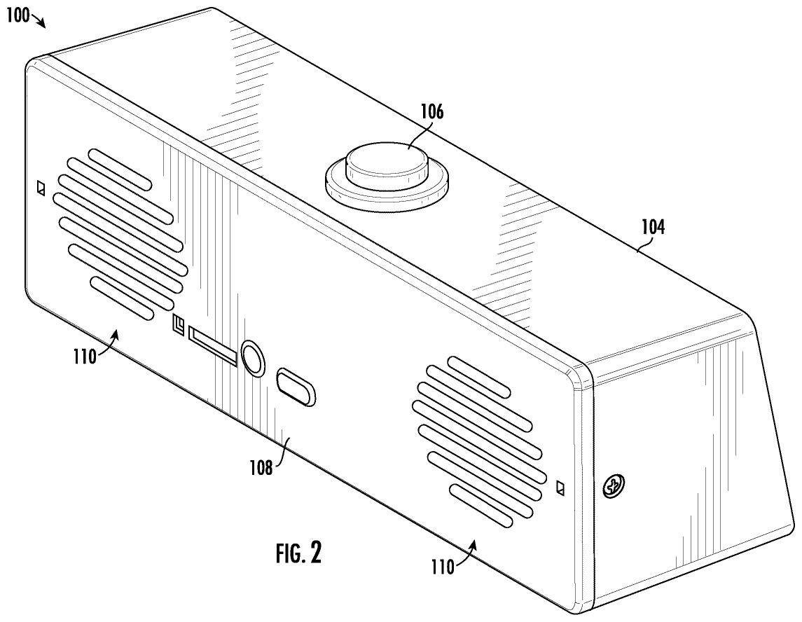

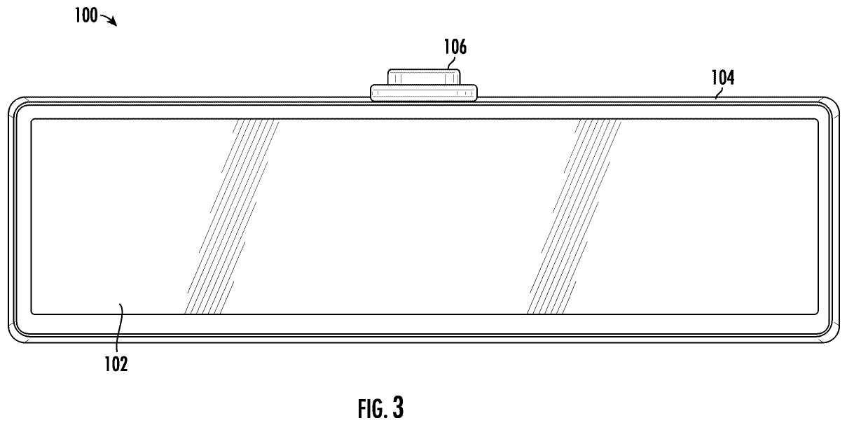

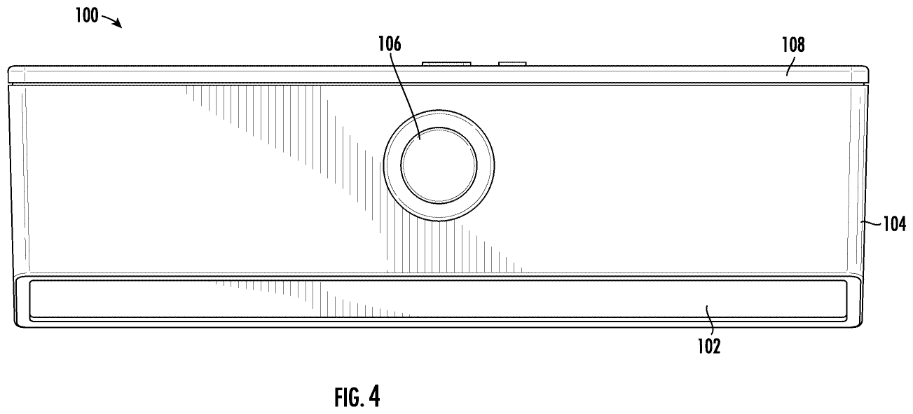

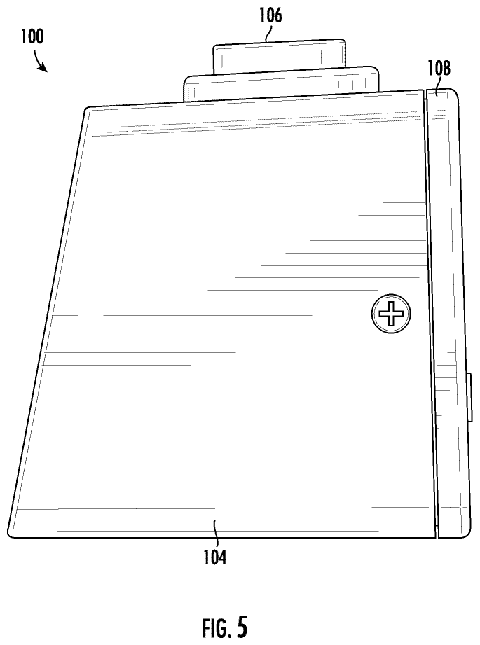

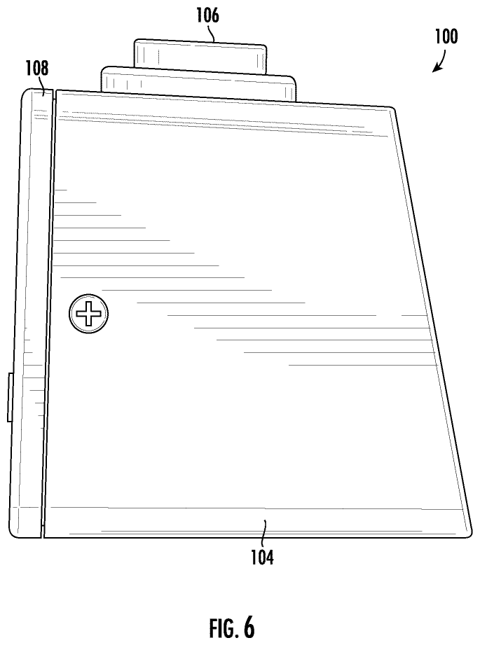

One example notification device is illustrated in FIGS. 1-6, with FIG. 1 being a front, perspective view, with FIG. 2 being a rear, perspective view, with FIG. 3 being a front view, with FIG. 4 being a top view, with FIG. 5 being a right side view, and with FIG. 6 being a left side view. The notification device 100 may be a dedicated and standalone hardware device that operates separately from any tracking device itself and separate from any phone (or other user device). The notification device 100 may display blood glucose levels for one or more tracked individuals. The notification device 100 comprises a screen 102 where information may be presented, and the information that may be presented on the screen 102 is described in greater detail herein. While the notification device 100 comprises a screen 102, other notification devices may be provided without a screen, and information about a tracked individual's blood glucose levels or health status may be presented in other ways. For example, this information may be presented via a buzzer, an indicator light, a speaker, a projector, etc.

The notification device 100 also comprises an outer shell 104 that protects the inner contents of the notification device 100, with electronics positioned within the outer shell 104. A back cover 108 may be removably attachable to the outer shell 104, with the back cover 108 extending all the way along one side of the notification device 100. The back cover 108 may be removably attachable so that the inner contents of the notification device 100 may be added, removed, cleaned, or otherwise maintained. Additionally, as illustrated in FIG. 2, the back cover 108 may define openings 110 to provide ventilation for the inner contents of the notification device 100. The openings 110 may also be configured to allow sound to easily exit the notification device 100 through the openings 110.

A button 106 is also included, with the button 106 extending upwardly through the outer shell 104. The button 106 may be pushed to snooze or turn off alerts and to input other information to the notification device 100. While only one button 106 is illustrated for the notification device 100, other embodiments may include multiple buttons. In some embodiments, button 106 or another button may be pressed to cause multiple notification devices and/or other devices to be snoozed at one time (as used herein “snooze” may refer to causing the alert to be temporarily disabled and/or canceled). In some embodiments, pressing of a button on the notification device 100 in a certain manner (e.g., for a certain duration of time, in a certain tapping pattern, etc.) may cause snoozing of alerts being generated at that notification device 100 and/or at other devices (e.g., at another notification device, at cell phone of a parent, friend, etc., or at a tablet, a computer, etc.).

In some embodiments, the notification device 100 may be configured to provide remote alerts. With the remote alerts, the button 106 and other buttons described herein may be activated for an extended period of time, and this may cause the notification device to generate an alert signal that may be sent to another device or to indicate that there is a medical emergency. For example, the user (e.g., a tracked individual) may press the button 106 and hold down on the button 106 for about 1 second or more, about 3 seconds or more, about 5 seconds or more, or for another threshold period of time. However, in some embodiments, a button may simply be activated to generate the alert signal. Where this is the case, a cover may be added in some embodiments over the button to prevent the button from inadvertently being pressed.

The ability to provide remote alerts provides additional safety benefits for the notification device. If a tracked individual's blood glucose level gets really low, the blood glucose level may become so dangerous that the tracked individual may lose consciousness. When the person is in this state, functioning at all may be difficult. If the person is in this state, the person may still be able to press and hold the button 106 or issue a voice command. By pressing the button 106 or by issuing a voice command, an alert signal may be sent to the notification device 100 or another similar notification device, to a user device (e.g., a phone, a receiver, tablet, etc.) of another person or persons so that the individual being tracked may get assistance. In some embodiments, the notification device 100 may send a command using the internet that gets routed to another device, and this may be done through message queuing telemetry transport (MQTT) or other known internet messaging protocols. Once the alert signal is received at the other device, the recipient may receive a unique alarm that obtains the attention of the recipient. Additionally, other important information may be presented to the recipient such as the location of the tracked individual that is in danger, the name of the tracked individual, the name or some identification of the device, the blood glucose level for the tracked individual, the potential medical risks to the tracked individual (e.g., low blood glucose level, potential loss of consciousness, among others). However, further information may be presented, different information may be presented, or none of this information may be presented to the recipient in some embodiments. In some embodiments, signals may be sent to multiple receiving devices (e.g., to a loved one and to local authorities to obtain medical assistance). The ability to generate a remote alert also provides an extra layer of safety in the event an alert is not automatically generated for some reason. For example, readings from tracking devices may, at times, be inaccurate due to a variety of causes, such as dehydration. Despite the fact that an actual blood glucose level may be at a dangerous level, inaccurate readings may prevent alerts from being triggered because the readings may errantly indicate the tracked individual is in a normal range. Most tracked individuals are capable of feeling when their blood glucose is low and triggering the remote alert for assistance. The remote alert to a user's device may be the only alert that the user receives.

In some embodiments, the notification device may be configured to receive out-of-range indications from the tracking device that the blood glucose level either falls inside or outside of a desired range, and these out-of-range indications may not include a specific blood glucose level. These out-of-range indications may be sent at a greater frequency than other indications that include a specific blood glucose level. For example, indications having specific blood glucose level may be sent every five minutes, but out-of-range indications may be sent every minute and/or whenever they occur. The use of both out-of-range indications and indications with specific blood glucose level may be beneficial to optimally use the information made available by tracking devices. In this regard, some tracking devices are configured to track whether blood glucose levels are out-of-range at a higher frequency (e.g., once a minute) than the frequency at which the tracking devices track specific blood glucose levels (e.g., once every five minutes). In some embodiments, if an indication is sent indicating that the blood glucose level is out of range, then the notification device or another device may be configured to cause another indication to be generated indicating the specific blood glucose level for the tracked individual-however, if an indication is sent indicating that the blood glucose level is within range, then no additional indication may be sent regarding the specific blood glucose level at that time.

Additionally or alternatively, the notification device 100 may include other user interfaces so that the user may input data or cause changes in the operation of the notification device 100. In some embodiments, the notification device 100 may be connected to a phone or another user device through an application on the phone or the user device, and the user may input data or cause changes in the operation of the notification device 100 using the application on the phone. For example, the user may snooze an alert for one or more notification devices at the user's phone. Where this is the case, the user may, for example, be a parent who wishes to monitor blood glucose levels for two of the parent's children—when multiple notification devices have their alarms going off, the parent may press a button to snooze one of the alerts or to snooze multiple active alerts.

Additionally, in some embodiments, the notification device 100 may be configured to provide vibration alerts. With the vibration alerts, the notification device 100 or a vibration device within the notification device 100 or externally connected to the notification device 100 may vibrate to get the attention of the user. In some embodiments, vibration alerts may be provided with randomized audio and vibration patterns to help prevent brain habituation. For example, the alerts may be provided in a first pattern on a first occasion, a second pattern on a second occasion, and a third pattern on a third occasion, with each of the patterns being different from each other. In some embodiments, the audio and/or vibration pattern may be randomly generated on each occasion or selected from a group of available patterns on each occasion, and the group of available patterns may have two or more patterns, three or more patterns, five or more patterns, ten or more patterns, etc. Where audio and/or vibration patterns are randomly generated, the length of any audio or vibration may be adjusted, the pitch or magnitude of audio or vibration may be adjusted, the duration of silence between audio notes or periods with vibration may be adjusted. Even within a single pattern, these parameters may be changed (e.g., audio or vibration may be generated for 5 seconds, pause for 1 second, and may be generated again for only 2 seconds). Patterns may be different from each other in terms of the magnitude of vibration, the duration of time that vibration occurs, the duration of time that vibration does not occur, the frequency of vibration, the frequency of instances where vibration does not occur, the noise level of any audio that is generated, the audio source, the duration of time that audio is played, the duration of time that audio is not played, the frequency at which audio plays, the frequency of instances that audio does not play, etc.

In some embodiments, the alert volume may be incremented so that the volume becomes incrementally louder over time. Alternatively, the first instance of an alert may simply trigger a vibration alert, and subsequent instances of an alert may trigger an audio alert and/or a vibration alert, with the alerts escalating in magnitude. Thus, if the user fails to address the alerts that are generated, the alerts may continue to get louder or may otherwise have a greater magnitude until the user addresses the issue or disables the alerts to indicate that the issue has been resolved. In some embodiments, the alerts may become louder or may have a greater magnitude after each minute that the alert is not snoozed, but the alerts may increase in volume or magnitude in different time intervals (e.g., once every 5 seconds, once every 15 seconds, once every 30 seconds, once every 2 minutes, once every 5 minutes, etc.).

In some embodiments, the user may receive an emergency communication if the user(s) do not address an alert within a specified time. This emergency communication may be a call or a text to a designated number, or the emergency communication may be an email sent to a designated email address. The user may be able to adjust the settings in some embodiments to enable or disable emergency communications, to enable or disable emergency communications at certain time windows, to change the time before any emergency communication is made, or to automate the emergency communication if an alert has not been snoozed within a certain time. In some embodiments, the notification device may be configured to make an emergency communication to another device if the alert has not been snoozed within a certain time, and this emergency communication may be provided in the form of a phone call, a text message, an email, a notification, etc.

In some embodiments, snoozing alerts may be generated on other devices that are remote from the notification device 100. For example, alerts may be generated on a smart phone, a receiver, a tablet, a computer, or another device, with signals being sent via a wireless connection in some embodiments. Signals may be sent via low range wireless communication approaches such as Bluetooth, Bluetooth low energy (BLE), Wi-Fi, etc. However, signals may be sent using other wireless communication technologies. In some embodiments, signals may be sent via wired connections between the notification device and other devices. For example, the notification device may be connected via a wired connection to a smart phone, a receiver, a tablet, a computer, etc., and a snoozing alert may be generated at the tablet or the computer.

In some embodiments, alerts may be provided to indicate when no data is available or where limited data is available. This may, for example, occur when local outages occur, when the continuous glucose monitor or another tracking device is out of power or is disconnected, when the communication channel for data is disconnected (e.g., when Bluetooth, Wi-Fi, etc. are disconnected), or when other issues arise that cause data to be unavailable. In instances where the communication channel for data is disconnected, data may still continue to be collected by the tracking device so that the data may be uploaded to a notification device or to the smart phone of the user.

In some embodiments, distributed outage detection may be provided to users to indicate when large-scale outages are occurring. For example, when one or more products or services provided by a third-party manufacturer (e.g., Dexcom®, FreeStyle Libre®, etc.) are down, indications may be provided to users to inform them of the issue. These indications may be provided at the notification devices, at another user device, or at another location. In some embodiments, the indications associated with distributed outage detection may be provided at a website that the user may access using his or her user device, and the indications may provide detailed information regarding the problem and the status of any troubleshooting. These distributed outage detection indications may inform the user that the problems are not related to any hardware or software issues with his or her particular devices, giving the user clarity regarding the problem as well as peace of mind. By providing distributed outage detection indications at a website, the traffic level at the website may be increased, which may lead to increased visibility for the products and which may potentially lead to increased product sales.

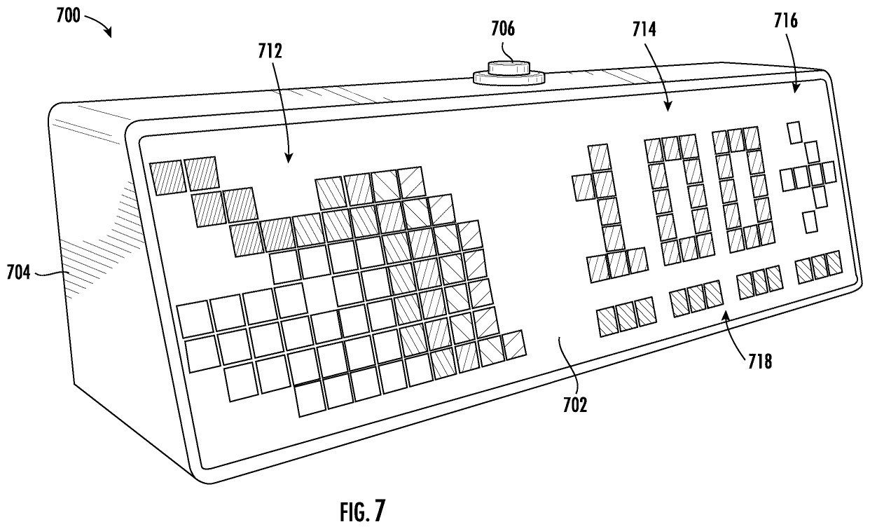

Another notification device 700 is illustrated in FIG. 7 with information presented on the screen 702 of the notification device 700. The notification device 700 may operate similarly to other notification devices described herein, and the notification device 700 may include an outer shell 704 and a button 706 similar to the notification device 100 of FIGS. 1-6.

The notification device 700 may display blood glucose levels for tracked individuals. The notification device 700 illustrates a numerical indicator 714, with this numerical indicator 714 being the number one hundred. This numerical indicator 714 is representative of the blood glucose level for the tracked individual, with the level being stated in milligrams per deciliter.

In the embodiment illustrated in FIG. 7, an indication 712 is illustrated on the screen 702. This indication 712 is representative of the blood glucose level for the tracked individual, and the indication 712 may be provided in the form of an emoji, icons, a word, a phrase. However, the indication 712 may also be provided in other forms as well. In some embodiments, the indication 712 may be a phrase that is presented. For example, the phrase may be “practice makes perfect” when the blood glucose level is at or near a perfect blood glucose level, the phrase may be “if you know you know” when the blood glucose level is not in the ideal blood glucose level range, and the phrase may be “mistakes were made” when the blood glucose level is in a very poor range.

In some embodiments, users may select their desired settings and setup for the notification device so that the information presented on the screen of the notification device is tailored as desired for the user preferences. For example, the user may make adjustments to select whether the notification device presents blood glucose levels for one tracked individual or for multiple tracked individuals. Settings may be adjusted to input user specific information such as the ideal blood glucose level(s) and other ranges of blood glucose level(s) that are less than ideal. For example, settings may be adjusted to have a ranges of Urgent Low, Low, In Range, High, and Urgent High. Where such ranges are used, blood glucose levels of less than about 60 may fall within the Urgent Low category, blood glucose levels between about 60 milligrams per deciliter and about 70 milligrams per deciliter may fall within the Low category, blood glucose levels between about 70 milligrams per deciliter and about 180 milligrams per deciliter may fall within the In Range category, blood glucose levels between about 180 milligrams per deciliter and about 250 milligrams per deciliter may fall in the High category, and blood glucose levels above about 250 milligrams per deciliter may fall in the Urgent High category (although other range values are contemplated). Alternatively, settings may be adjusted where the tracked individual has an A+ grade, an A grade, an A− grade, a B+ grade, a B grade, a B− grade, a C+ grade, and so forth. The A+ grade may correspond to blood glucose levels within five milligrams per deciliter of the ideal blood glucose level, the A grade may correspond to blood glucose levels outside of the A+ range but within ten milligrams per deciliter of the ideal blood glucose level, and so forth. However, other ranges may be used, and ranges may be adjusted by the user in some embodiments. For example, a simple grading range of A, B, C, D, F may be used. Additionally, different units may be used such as millimoles per liter or another unit rather than milligrams per deciliter. In some embodiments, rather than using an alphabetical grading system, other symbols, slogans, or indicators may be used. For example, as illustrated in FIG. 7, when the tracked individual has a perfect or near perfect blood glucose level, an indicator 712 may appear with a unicorn to indicate a “unicorn” blood glucose level, and when the tracked individual has a very poor blood glucose level, other indicators 712 may appear like a Grinch, Scrooge, a dumpster fire, etc.

In some embodiments, the indicator 712 may be a user customized emoji, an icon, a slang term, a color, or some other indicator. For example, the indicator 712 may be an emoji of a person clapping when the blood glucose level is perfect or near perfect, and the indicator 712 may be a facepalm emoji when the blood glucose level is poor. As another example, the indicator 712 may be a slang term or slogan such as “as good as it gets” when the blood glucose level is perfect or near perfect, and the indicator 712 may be a slang term, slogan, or quote such as “not good enough” or “small daily improvements over time lead to stunning results.”

In some embodiments, the user may be able to create their own indicators (e.g., emojis, icons, slang terms, colors, etc.) or users may be able to select from a list of available indicators. However, in other embodiments, the indicators may be pre-set and may not be customizable by the user.

In some embodiments, the indicators 712 may be color coded. For example, the indicators may be green when the blood glucose level is perfect or near perfect, indicators may be yellow when the blood glucose level is good but less than perfect, indicators may be orange when the blood glucose level needs improvement, and indicators may be red when the blood glucose level is very poor. The user may adjust the ranges of blood glucose levels corresponding to each color in some embodiments. Alternatively, in some embodiments, the color may gradually change based on changes in the blood glucose level rather than having ranges of blood glucose levels associated with different colors.

In the notification device 700, the screen 702 illustrates a number 714, a trend arrow 716, and a symbol 718. The number 714 may be the blood glucose level for a tracked individual, and the number 714 may be presented differently depending on the blood glucose level and/or the rate of change for the blood glucose level. For example, in the notification device 700, the number 714 is presented in green text and in a relatively small font. The trend arrow 716 serves as an indicator for the rate of change for the blood glucose level. The trend arrow 716 is oriented horizontally to indicate that the blood glucose level remains relatively constant. Other symbols 718 are also included to present further information. The symbols 718 are indicative of the time remaining until the next blood glucose level data is received. For example, in the embodiment illustrated in FIG. 7, between 4 and 5 minutes have elapsed since the previous update, with each of the symbols 718 representing an additional minute of time since the previous update.

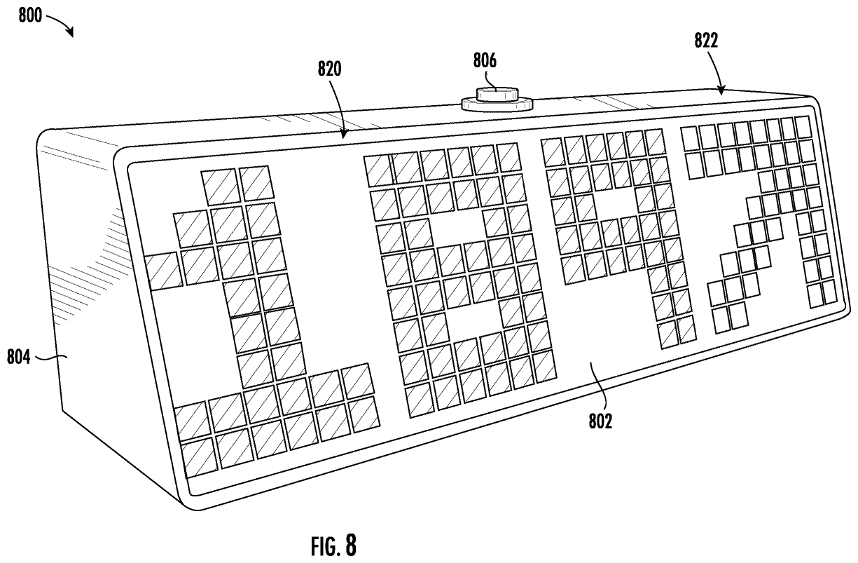

Another example notification device 800 is illustrated in the front, perspective view of FIG. 8. The notification device 800 may operate similarly to other notification devices described herein, and the notification device 800 may include a screen 802, an outer shell 804, and a button 806 similar to the notification device 100 of FIGS. 1-6. The screen 802 is presenting a number 820, with the number 820 indicating that the blood glucose level is about 189 milligrams per deciliter. Given the fact that the blood glucose level is relatively high and because the rate of change for the blood glucose level is increasing, the number 820 and the trend arrow 822 may be enlarged on the screen so that they are presented with greater emphasis, and the number 820 may be presented in the color pink to further emphasize the blood glucose level.

In some embodiments, a trend arrow 822 may be presented on the screen, with the trend arrow 822 depending on the rate of change or the delta for the blood glucose level. The trend arrow 822 may indicate how fast the blood glucose level is dropping or rising. The trend arrow 822 may be beneficial to give a user a better understanding of the tracked individual's blood glucose levels. Where the trend arrow 822 indicates a sharp increase in the blood glucose level and the blood glucose level is already relatively high, then this may be indicative of potential issues. Additionally, where the trend arrow 822 indicates a decrease in the blood glucose level and the blood glucose level is relatively high, then this may be indicative that the blood glucose level is returning to a desired level.

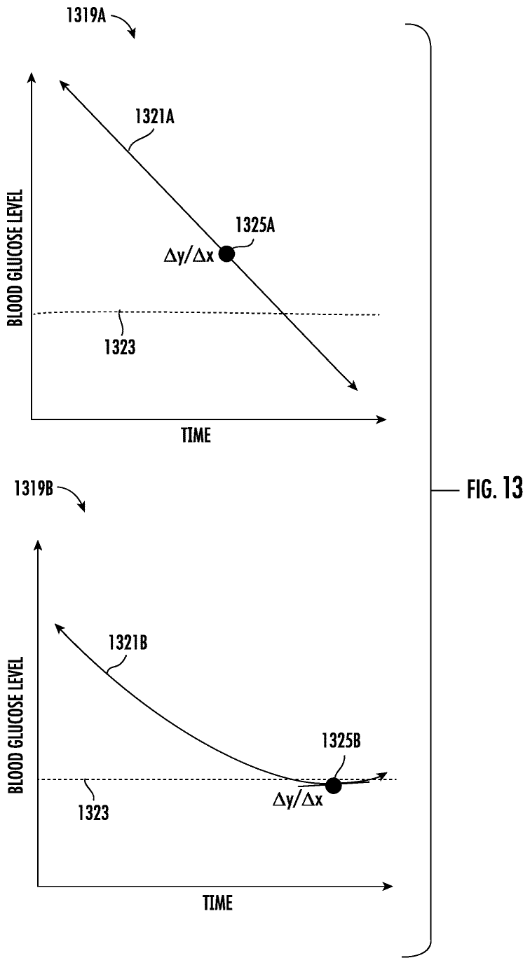

Turning ahead to FIG. 13, multiple graphs 1319A, 1319B are illustrated, with these graphs 1319A, 1319B illustrating the blood glucose level for tracked individuals over time. When the blood glucose levels are over the lower threshold 1323, the blood glucose levels may remain in a normal range. In some embodiments, the normal range may fall between an upper threshold and a lower threshold, but only a lower threshold 1323 is illustrated in the graphs 1319A, 1319B for the purposes of simplification.

In the graph 1319A, the plotline 1321A for the blood glucose level is initially in the normal range. However, the blood glucose level quickly approaches the lower threshold 1323, with the blood glucose changing at a constant rate (i.e., first derivative as a function of time). Given the relatively high rate of change (in terms of absolute value for the rate of change), an alert may be generated even before the blood glucose level falls below the lower threshold 1323. For example, an alert may be generated at a time corresponding to point 1325A. By considering the first derivative and/or the second derivative of the blood glucose level as a function of time in making alerts, alerts may be made earlier to help address issues swiftly. While the graph 1319A illustrates an alert being generated when the blood glucose level approaches a lower threshold for a normal range for the blood glucose level, an alert may similarly be generated where the blood glucose level approaches an upper threshold of a normal range for the blood glucose level.

Additionally, in some embodiments, other factors such as dehydration levels or other health related factors may be considered in determining whether it is appropriate to generate an alert. Readings from tracking devices may, at times, be inaccurate due to a variety of causes such as dehydration as an example. So, while a patient may otherwise have a low blood glucose level that falls out of the normal range, an alert may not be triggered because the dehydration levels indicate that the determined blood glucose level is not accurate. Notification devices and/or other devices may receive data from other sensors or other sources to identify and consider these issues when evaluating blood glucose levels.

In the graph 1319B of FIG. 13, the plotline 1321B for the blood glucose level is initially in the normal range, and the blood glucose level is approaching the lower threshold 1323. Eventually, the blood glucose level dips below the lower threshold 1323 proximate to point 1325B. However, in the illustrated example of the graph 1319B, the rate of change Δy/Δx (i.e., the first derivative as a function of time) of the blood glucose level at the point 1325B actually indicates that the blood glucose level is increasing. Given the rate of change Δy/Δx at the time corresponding to the point 1325B, a notification device may determine that no alert should be generated even though the blood glucose level is below the lower threshold 1323. Thus, in the example illustrated in the graph 1319B of FIG. 13, the consideration of the first derivative and the second derivative of the blood glucose level as a function of time may increase the accuracy of alerts so that alerts are only generated when necessary.

In some embodiments, the notification devices may consider the delta change value in determining whether to generate an alert, the type of alert, or the timing of the alert. For example, when the delta change value is relatively low to indicate that the change in blood glucose level is changing gradually, alerts may be provided less frequently or the notification devices may hold off on providing an alert. As another example, where the delta change value indicates that the blood glucose level is decreasing rapidly and that the blood glucose level is already very low, then alerts may be provided with high urgency, and alerts may be provided frequently.

Additionally, a delta threshold may be set. For example, the delta threshold may be set to around 5 milligrams per deciliter for a time thirty minutes, but other delta threshold values and corresponding times may be used, and the delta threshold and corresponding times may be customizable by the user in some embodiments. Where a delta threshold value is used, the blood glucose level may extend out of a specified range for a certain period of time. For example, a low threshold alert may be set to 70 milligrams per deciliter with a delta threshold of 5 milligrams per deciliter for 30 minutes. The alert may be ignored as long as the blood glucose level is higher than 65 milligrams per deciliter (the 70 milligrams per deciliter low threshold alert, minus the delta of 5 milligrams per deciliter). If the blood glucose drops below the allowed delta total of 65 milligrams per deciliter, the alert may immediately trigger. Or if the blood glucose level is still under 70 milligrams per deciliter after 30 minutes, the alert may be triggered. However, if the blood glucose level only extends into the blood glucose level range of 65 to 70 milligrams per deciliter for less than thirty minutes and then increases back above 70 milligrams per deciliter, then no alert may be triggered.

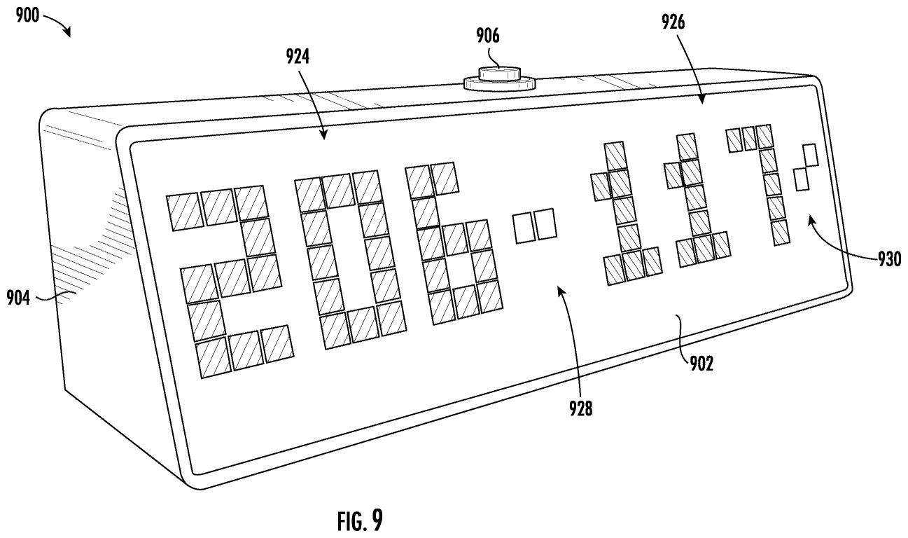

Another example notification device 900 is illustrated in the front, perspective view of FIG. 9. The notification device 900 may operate similarly to other notification devices described herein. In the example embodiment illustrated in FIG. 9, the notification device 900 displays blood glucose levels on the screen 902 for multiple people at same time. On the screen, a first number 924 is presented for a first tracked individual, with the first number 924 indicating that the blood glucose level for the first tracked individual is about 206 milligrams per deciliter. Additionally, a second number 926 is presented for a second tracked individual, with the second number 926 indicating that the blood glucose level for the second tracked individual is about 117 milligrams per deciliter. Additionally, a first trend arrow 928 is illustrated on the screen 900 to indicate that the blood glucose level for the first tracked individual is remaining approximately constant, and the second trend arrow 930 is illustrated on the screen 900 to indicate that the blood glucose level for the second tracked individual is increasing. The notification device 900 may include a screen 902, an outer shell 904, and a button 906 similar to the notification device 100 of FIGS. 1-6. The first number 924 is presented in pink, and the second number 926 is presented in green. These colors may be indicative of the tracked individuals so that the tracked individuals or other users (e.g., parents) may remember which number is associated with which tracked individual. Alternatively, the colors may be indicative of whether or not the blood glucose levels fall within the ideal range or farther away from the ideal range.

Where notification devices are configured to present information for multiple tracked individuals at the same time like notification device 900, the notification device may be configured to cause alerts to be generated for each of the tracked individuals. Additionally, where notification devices are used to track multiple tracked individuals, then the alerts that are generated for the notification devices may be different depending on the tracked individual. For example, a first alert may be generated when the blood glucose levels are out of range for a first tracked individual but not the second tracked individual, a second alert may be generated when the blood glucose levels are out of range for a second tracked individual but not the first tracked individual, and a third alert may be generated when the blood glucose levels are out of range for both tracked individuals. In this example, each of the first, second, and third alerts may be different from each other to allow users to determine the tracked individual(s) that the alert applies to. The alerts may be different in terms of the sound that is generated by the notification device, but the alerts may also be generated by flashing the information on the screen or otherwise changing the presentation on the screen of the notification device.

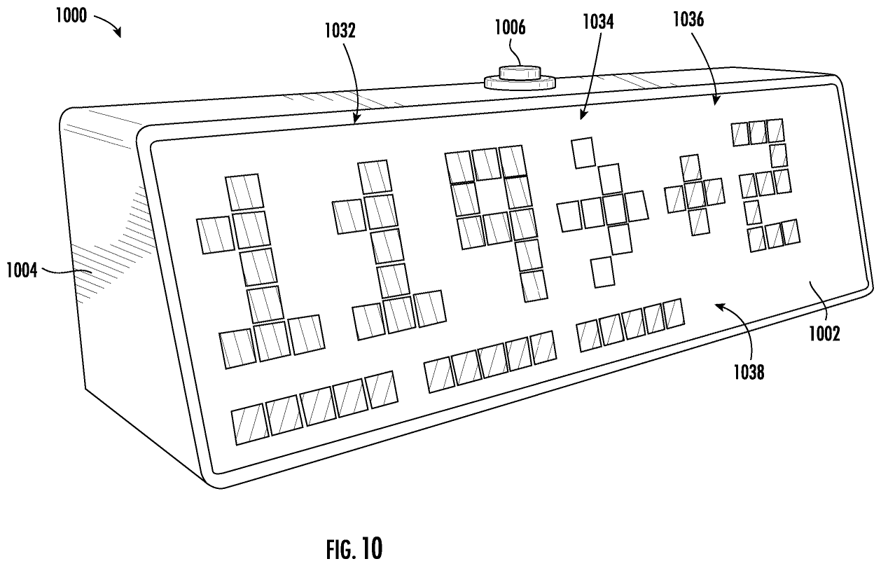

FIG. 10 is a front, perspective view illustrating an example notification device 1000 having a screen presenting different information. The notification device 1000 may operate similarly to other notification devices described herein. The notification device 1000 may include the screen 1002, an outer shell 1004, and a button 1006 similar to the notification device 100 of FIGS. 1-6.

On the screen 1002 of the notification device 1000, a number 1032 may be presented to indicate the blood glucose level for the tracked individual. The number 1032 indicates that the blood glucose level is about 119 milligrams per deciliter, and the number 1032 is presented in a green color. The screen 1002 also includes a trend arrow 1034 to indicate that the blood glucose level is relatively constant, and the indicator 1036 may show an indication of the change in blood glucose level over some duration of time. For example, the indicator 1036 indicates that the blood glucose level has changed by about 2 milligrams per deciliter over the previous minute. Additional symbols 1038 may also be included to present further information. For example, the symbols 1038 may represent the amount of time that has elapsed since the displayed reading, with each bar representing a minute that has elapsed. Thus, in the illustrated notification device 1000, the screen 1002 indicates that the 119 milligrams per deciliter reading was about 3 minutes ago. The individual pixels in the bars may represent subdivisions of a minute. For instance, each pixel may represent a twelve second increment and the five pixels in the bar total sixty seconds or one minute. The major continuous glucose monitors (CGMs) today update readings between every 1 minute and every 5 minutes. When a low blood glucose event happens, the amount of time from the previous reading may be critical information to know when to expect the next reading to see if you have had enough carbohydrates to correct the low glucose.

In some embodiments, the notification device 1000 or other notification devices described herein may be configured to present blood glucose levels over a certain period of time. For example, the notification devices may be configured to present a blood glucose level over a 1 minute interval, a 2 minute interval, a 5 minute interval, a 10 minute interval, a 15 minute interval, a 30 minute interval, a 60 minute interval, a three hour interval, a six hour interval, a twelve hour interval, a 24 hour interval, or some other interval. In some embodiments, the notification device 1000 or other notification devices described herein may be configured to present a daily time in range value for users. This daily time in range value may be the actual or approximate amount of time that the blood glucose levels for the user are within certain ranges. In some embodiments, these ranges may simply be a desired range and other ranges that fall outside of the desired range (e.g., above desired range, below desired range). The desired range may be preset in some embodiments without requiring user input, but the desired range may be adjusted in some embodiments to tailor the desired range based on the user's preferences. In some embodiments, more than three ranges may be monitored. For example, the ranges may include an extremely low blood glucose level range, a low blood glucose level range, a desirable blood glucose level range, a high blood glucose level range, and an extremely low blood glucose level range. In some embodiments, the ranges may be preset without requiring user input, but the desired range may be adjusted in some embodiments to enable a user to tailor the desired range based on the user's preferences.

In some embodiments, the user may receive a score based on the user's current blood glucose level, based on the user's most recent daily time in range values, and/or based on the user's daily time in range values over the course of the past several days, week(s), or month(s). The score may be presented in the form of the average or median daily time in range value in some embodiments, but the score may be provided in some other form such as an alphabetical score (A+, A, A−, B+, C, D, F, etc.), a numerical score (e.g., 1 to 100), etc. The score may be presented on a notification device 1000 or another notification device described herein. In some embodiments, the presentation on a screen of a notification device 1000 may be altered based on the user's score. For example, when the user's score is good, an emoji (e.g., a unicorn to represent a “unicorn” score), customizable term or slang term, or some other material may be presented to indicate that the score is good.

In some embodiments, material may be emphasized on the screen. Emphasis may be used to indicate when scores are good, but emphasis may also be used when blood glucose levels are poor and action is necessary to improve the blood glucose levels. For example, emphasis may be caused by flashing indicators or other information presented on the screen, by changing the color of the background of the screen, the text on the screen, the font size on the screen, the font of the text on the screen, etc. By providing emphasis, those with hearing impairments may be adequately alerted if any issues arise, and they may also have peace of mind that no issues are present even when alerts are not being presented.

In some embodiments, notification devices may be configured to adjust their appearance based on the time of day. For example, the notification devices may be connected to a clock to receive clock information, and the background color, the brightness of the screen, the color of certain text or indicators on the screen, or the amount of information presented on the screen may be adjusted based on the time of day. For example, during the evening during times when the user is likely sleeping, the notification devices may be changed to a night mode where the screen has a darker background, where text and indicators have reduced contrast, and/or where less information is presented on the screen. By contrast, during the day during times when the user is likely awake, the notification devices may be used in a normal mode where the screen has a brighter background, text and indicators that are brighter in color or that have more contrast, and/or more information presented on the screen. In some embodiments, when an alert is generated, the brightness, contrast, and/or presentation for material on the screen may be different even when the notification devices are operating in a night mode.

In some embodiments, the notification device may be presented in sleep mode at night, with no material presented on the screen. In some embodiments, the notification device may be configured so that it does not present any material on the screen during normal sleeping hours when in sleep mode and when the tracked individual's blood glucose levels are in a desired range, and the notification device may be configured so that it will present material on the screen during normal sleeping hours when in sleep mode and when the tracked individual's blood glucose levels are not in a desired range. The sleep mode may assist in reducing the energy consumption of the notification devices and may help to reduce distractions to the user as the user is trying to fall asleep. The sleep mode may also be used at other times of day, and the sleep mode is not limited to being used during normal sleeping hours in some embodiments.

In a similar vein, notifications may also be adjusted in operation based on the time of day. For example, alerts may be enabled at certain times of day, and alerts may be disabled at certain times of day. The alert schedule may be set with times where alerts are enabled and disabled. The alert schedule may be preset by a manufacturer in some embodiments. The alert schedule may be set by default to generate alerts at all times of day when the notification devices are first sold. However, the alert schedule may be customizable by the user in some embodiments. Where the alert schedule is customizable by the user, the user may customize the alert schedule in hour increments, 30 minute increments, 15 minute increments, 10 minute increments, five minute increments, or one minute increments in some embodiments. However, the increments that the alert schedule may be customized in may be different in other embodiments. By allowing for modification of the alert schedule, the times may be easily adjusted when they may be less needed. For example, when the user is a child, a parent may adjust the times of operation so that the alerts are turned off when the child is typically at home during the day, and the alerts may be turned on at other times (e.g., when the child is at school, at a sporting event, when the child is asleep).

In some embodiments, notification devices may be used to display insulin pump data. For example, data may be presented to indicate whether the insulin pump is working appropriately, the battery level for the insulin pump, the amount of insulin on board, recent insulin doses, other data regarding the insulin pump.

Various features may be included to give the notification devices a selected theme. For example, the notification devices may be Halloween themed, Christmas themed, Easter themed, Thanksgiving themed, etc. However, the notification devices may also have other themes (e.g., superhero theme, sports theme, cartoon themed, etc.). The notification devices may include a projection device that is configured to project images towards a ceiling or towards another surface. The projection device may generate a single color, an image, a symbol, a number, etc., and the material that is generated may be dependent on the blood glucose level of the tracked individual. The projection device may generate themed images (e.g., northern lights images, Halloween theme, Christmas theme, Easter theme, Thanksgiving theme, etc.). A selected theme may also be implemented through the use of themed sounds (e.g., happy Christmas music or Christmas carols, scary music for Halloween). However, selected themes may be implemented through other ways such as the brightness of images presented, the color presented, the symbols presented, the typeface used for any text or numbers that are presented, and the like.

In some embodiments, the notification device may have connectivity to other lights such as lights on a Christmas tree, with the lights being configured to change in color. The color of the light that is emitted from the lights may be dependent on the blood glucose level of the tracked individual(s). For example, lights may be green when the blood glucose levels are good, yellow when blood glucose levels are in a moderate level, and red when the blood glucose levels are poor. In some embodiments, the notification device may cause the lights to flash repeatedly to alert the user(s) if the blood glucose level is poor or very poor.

The notification devices described herein may be used in a variety of different settings. For example, the notification devices may be utilized in a bedroom as an alarm clock or adjacent to an alarm clock, in an office, in a living room, in a workspace, a campsite, a recreational vehicle, or in a vehicle as a car dashboard device. However, the notification devices may also be used in other locations. The notification device may be configured to operate as an alarm clock.

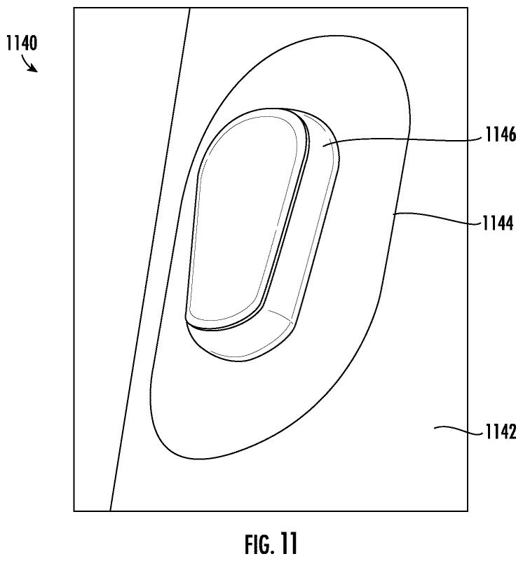

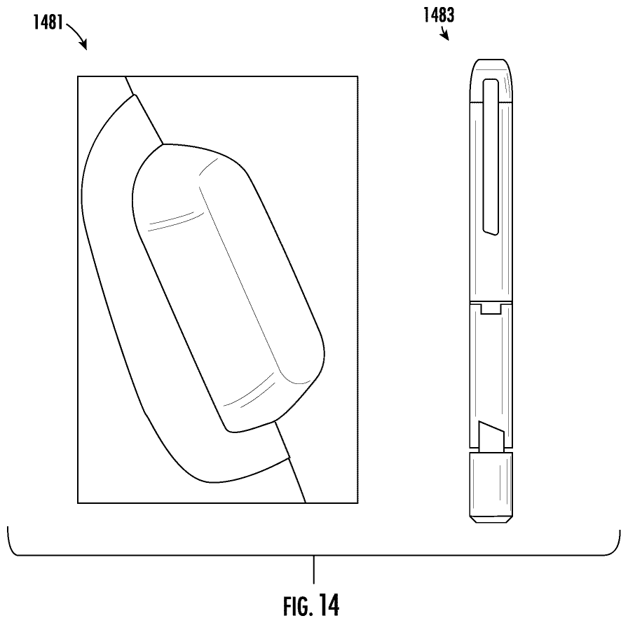

FIG. 11 illustrates a tracking device in the form of a continuous glucose monitor 1146 attached on an arm 1142 of a tracked individual 1140. The continuous glucose monitor 1146 may be attached using an attachment mechanism 1144, which may include an adhesive material. The continuous glucose monitor 1146 may obtain blood glucose level data for the tracked individual 1140, and the data from the continuous glucose monitor 1146 may be communicated to a smart phone for use on an application so that the data may then be communicated from the smart phone to the notification device for display. However, in some embodiments, a direct communication link may exist between the continuous glucose monitor 1146 and the notification devices described herein, or data from the continuous glucose monitor may be communicated to the notification device in other ways. FIG. 14 also illustrates an example of an insulin pump 1481 and an insulin pen 1483. The insulin pump 1481 may be configured to hold insulin and may include a cannula that goes under the skin to deliver insulin to the person wearing the insulin pump 1481. The insulin pump 1481 may be configured to send data related to the operation of the insulin pump 1481 to other devices. For example, the insulin pump 1481 may be sent to another device so that data associated with the insulin pump 1481 may be displayed. The insulin pen 1483 may be a smart insulin pen that is configured to track insulin doses, and the insulin pen 1483 may be configured to send this insulin dose data to other devices such as to a screen so that the insulin dose data or other data based on the insulin dose data may be displayed.

While various embodiments described herein are related to notification devices for monitoring blood glucose levels, it should be understood that the notification devices may additionally or alternatively be used to track other types of data or health data. For example, the notification devices may be used to track and present information related to a heart rate, a blood pressure, a distance traveled, a number of steps, a number of calories burned or consumed, the amount of carbohydrates consumed by the tracked individual during a meal or during a day, etc.

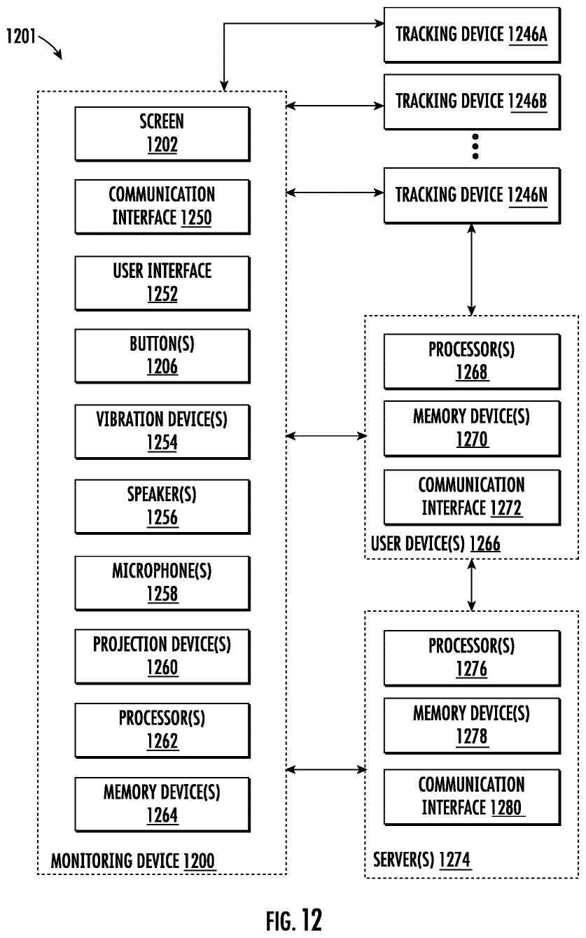

FIG. 12 illustrates a block diagram with various components of a system 1201 for operating the notification device 1200. The system 1201 may comprise a notification device 1200, a plurality of tracking devices, one or more user devices 1266, and one or more servers 1274.

The user device(s) 1266 may include one or more processors 1268, one or more memory devices 1270, and a communication interface 1272. The user device(s) 1266 may be provided in the form of a computer, a cell phone (e.g., a smart phone), a tablet, a receiver, a display, etc. In some embodiments, data may be driven to other user device(s) 1266 for use with other software or applications like Nightscout™, Gluroo™, etc. The system 1201 may also include one or more servers 1274. The server(s) 1274 may include one or more processors 1276, one or more memory devices 1278, and a communication interface 1280. These components of the user device(s) 1266 and the server(s) 1274 are described in greater detail herein.

The notification device 1200 includes a display in the form of one or more screens 1202. The screen(s) 1202 may be configured to present images and may include or otherwise be in communication with buttons and/or a user interface 1252 configured to receive input from a user. The screen(s) 1202 may be, for example, a conventional LCD (liquid crystal display), a touch screen display, or any other suitable display known in the art upon which images may be displayed. However, in some embodiments, no screen 1202 is provided. Where a notification device is provided without any screen, a power light or some other indicator may be included to indicate that the notification device is on or to indicate a state of the notification device. Alternatively, a notification device without a screen may have a blinking light where the blink indicates that the notification device is operating correctly, and the color of the light may change based on the blood glucose level.

The notification device 1200 may also include a user interface 1252. The user interface 1252 may include, for example, a keyboard, keypad, function keys, buttons, a mouse, a scrolling device, input/output ports, a touch screen, or any other mechanism by which a user may interface with the system. The notification device 1200 may also include one or more buttons 1206. The button(s) 1206 may be in the form of a push button or another actuatable mechanism where the user(s) may activate the button(s) 1206 to input information for use at the notification device 1200. For example, the button(s) 1206 may be activated to snooze an alarm on the notification device 1200 or to input other information. The notification device 1200 also includes a communication interface 1250. The communication interface 1250 is discussed in greater detail herein.

The notification device 1200 may also include one or more vibration devices 1254. The vibration device(s) 1254 may be a buzzer or some other device that generates vibration within the notification device 1200 or external to the notification device 1200. Vibration device(s) 1254 may be activated to generate vibration for an alarm. The vibration device(s) 1254 may be configured to generate vibration in differing or even random ways to prevent users from experiencing brain habituation.

In some embodiments, the notification device 1200 may comprise one or more speakers 1256. The speaker(s) 1256 may be used to provide audio alerts. Additionally, the speaker(s) 1256 may provide blood glucose levels in audio form for those with visual impairments, and the blood glucose levels may be converted from text or numbers to audio that is generated at the speaker(s) 1256. The audio alerts may be in the form of generic sounds (e.g., clapping), themed sounds (e.g., happy Christmas music), etc.

The notification device 1200 may also include one or more microphones 1258. The microphone(s) 1258 may be configured to receive voice commands from the user(s) or others. For example, the voice commands may be used to change the settings for the notification device 1200 so that the brightness level is changed, so that user information is changed, so that the color scheme is changed, to activate an emergency notification to a remote device, and to make other changes. However, in some embodiments, no microphone is included in the notification device 1200.

The notification device 1200 may also include one or more projection devices 1260. The projection device(s) 1260 may be configured to project images towards a ceiling or towards another surface. The projection device(s) 1260 may generate a single color, an image, a symbol, a number, etc., and the material that is generated may be dependent on the blood glucose level of the tracked individual. For example the projection device(s) 1260 may be configured to generate themed images (e.g., northern lights theme, Christmas theme, etc.)

The notification device 1200 also includes one or more processors 1262. The processor(s) 1262 may be configured to perform a variety of functions, and the processor(s) 1262 may execute operations or instructions stored at the memory device 1264 or at another location. Processor(s) 1262 may cause presentation of images, information, symbols, etc. on the screen 1202. Processor(s) 1262 may be configured to receive data from a tracking device or another device, cause the blood glucose levels or other health information to be presented on the screen, and cause any other representations associated therewith to be changed in approximately real-time. Processor(s) 1268 of the user device(s) 1266 and processor(s) 1276 of the server(s) 1274 may be configured to execute various instructions stored in other memory devices.

The processor(s) 1262, 1268, 1276 may be any means configured to execute various programmed operations or instructions stored in a memory device (e.g., memory devices 1264, 1270, 1278) such as a device or circuitry operating in accordance with software or otherwise embodied in hardware or a combination of hardware and software (e.g. a processor operating under software control or the processor embodied as an application specific integrated circuit (ASIC) or field programmable gate array (FPGA) specifically configured to perform the operations described herein, or a combination thereof) thereby configuring the device or circuitry to perform the corresponding functions of the processor(s) 1262, 1268, 1276 as described herein.

The notification device 1200 also includes memory device(s) 1264. Similarly the memory device(s) 1270 may be provided in the user device(s) 1266 and the memory device(s) 1278 may be provided in the server(s) 1274. In an example embodiment, the memory devices 1264, 1270, 1278 may include one or more non-transitory storage or memory devices such as, for example, volatile and/or non-volatile memory that may be either fixed or removable. The memory devices 1264, 1270, 1278 may be configured to store instructions, computer program code, health data, location data, and additional data in a non-transitory computer readable medium for use, such as by the processor(s) 1262, 1268, 1276 for enabling the components of the system 1201 to carry out various functions in accordance with example embodiments of the present invention. For example, the memory devices 1264, 1270, 1278 may be configured to buffer input data for processing by the processor(s) 1262, 1268, 1276. Additionally or alternatively, the memory devices 1264, 1270, 1278 may be configured to store instructions for execution by the processor(s) 1262, 1268, 1276. The memory devices 1264, 1270, 1278 may include computer program code that is configured to, when executed, cause processor(s) 1262, 1268, 1276 to perform various methods described herein. The memory devices 1264, 1270, 1278 may serve as non-transitory computer readable mediums having stored thereon software instructions that, when executed by one or more processors, cause methods described herein to be performed.

The communications interfaces 1250, 1272, 1280 may be configured to enable communication to other components of the system 1201. For example, as information is obtained from tracking device(s) 1246A-1246N, the information may be received at any of the communications interfaces 1250, 1272, 1280 so that the information may be stored for later use or so that the information may be presented on the screen 1202. In some embodiments, the information obtained from the tracking device(s) 1246A-1246N is sent first to the user device(s) 1266 and/or the server(s) 1274 for cloud storage before the information is sent to the notification device 1200.

The communications interfaces 1250, 1272, 1280 may also include one or more communications modules configured to communicate with one another in any of a number of different manners including, for example, via a network. In this regard, the communications interfaces 1250, 1272, 1280 may include any of a number of different communication backbones or frameworks including, for example, Ethernet, global positioning system (GPS), cellular, Wi-Fi, or other suitable networks. The network may also support other data sources, including GPS. In this regard, numerous other peripheral devices may be included in the system 1201. In some embodiments, some or all of the communications interfaces 1250, 1272, 1280, may be configured to communicate using short-range wireless technologies such as Bluetooth (e.g., Bluetooth Version 4.1 or another version), Wi-Fi, NearLink, near-field communication (NFC), low power wide area networks (LPWAN), ultra-wideband (UWB), wireless local area network (WLAN) in accordance with IEEE 802.11(b), IEEE 802.11(g), and/or IEEE 802.11(n) standards, and/or low-rate wireless personal access networks (LR-WPAN) pursuant to the IEEE 802.15.4 standard.

In the embodiment illustrated in FIG. 12, the system 1201 comprises N continuous tracking devices, with a first tracking device 1246A, a second tracking device 1246B, and additional tracking devices up to an Nth tracking device 1246N. In some embodiments, systems may be provided with only one tracking device, but two or more tracking devices may be used in other embodiments.

While the system 1201 is illustrated with various connections between components, these connections may be different in other embodiments. For example, there may be direct connections between the tracking device(s) 1246A-1246N and the server(s) 1274. Additionally, the connections between the components may be wireless connections. For example, the notification device 1200 may connect to other devices using a Bluetooth connection or another similar short range wireless technology. However, other connection approaches may be used for the notification device 1200. Components such as the server(s) 1274, the user device(s) 1266 and the tracking devices 1246A-1246N may be connected together in a wide variety of ways (e.g., Bluetooth, a cellular connection, another wireless connection, etc.). Connections may also be wired connections in some embodiments.

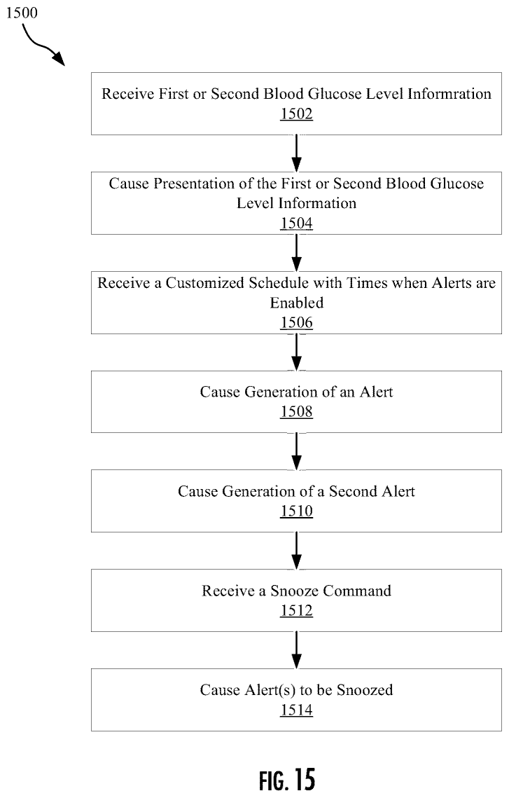

FIG. 15 illustrates an example flow chart with a method 1500 for presenting blood glucose level information for a tracked individual.

At operation 1502, first and/or second blood glucose level information may be received. The first blood glucose level information and the second blood glucose level information may each include at least one of a blood glucose level, a first derivative for the blood glucose level as a function of time, or a second derivative for the blood glucose level as a function of time. The first and second blood glucose level information may be received from a respective tracking device, with the tracking device being a continuous blood glucose monitor, an insulin pump, a smart insulin pen, or a similar device. Where first and second blood glucose level information are received, this information may be for two different tracked individuals (e.g., two children of a parent).

In some embodiments, the first and/or second blood glucose level information may include data having specific blood glucose level information. However, in some embodiments, the first and/or second blood glucose level information may also include indication that the blood glucose level either falls inside or outside of an acceptable range, with these indications not including any specific blood glucose level. These indications may be received at a more regular frequency than the frequency at which data having specific blood glucose level information is received, allowing alerts to be generated more quickly if a tracked individual is outside of an acceptable range.

At operation 1504, presentation of the first and/or second blood glucose level information may be caused. For example, presentation may be caused on a screen of a notification device or at another location.

At operation 1506, a customized schedule may be received with times when alerts are enabled. By doing so, alerts may only be generated during time periods when the user's customized schedule allows alerts, and alerts may be prevented during other time periods. However, in some embodiments, even when the user has disabled alerts during a particular time, alerts may still be generated if the tracked individual triggers the alert or if the blood glucose level information is extremely poor.

At operation 1508, generation of an alert may be caused. This may be caused based on the first and/or the second blood glucose level information, and the alerts may include audio alerts, visual alerts, vibratory alerts, or other similar alerts.

At operation 1510, generation of a second alert may be caused. The alerts may be randomized to prevent brain habituation—for example, the second alert that is generated may be distinct from the first alert (e.g., in its sound, color, frequency, etc.). In some embodiments, if a user has not snoozed an alert after a certain duration of time, a second alert having a greater magnitude may be generated. The magnitude may be greater in terms of volume level, brightness, flashing frequency, prominence of a visual alert display, or in other ways. In some embodiments, if a user has not snoozed an alert after a certain duration of time, a second alert may be caused that sends a notification to another device, with the notification including a phone call, a text message, an email, and/or a notification alert.

At operation 1512, a snooze command may be received. In some embodiments, this snooze command may be received at a separate device from the notification devices described herein, such as from a remote computer or phone. However, in some embodiments, snooze commands may simply be received at a notification device.

At operation 1514, snoozing of one or more alerts may be caused. In some embodiments, snoozing may occur only at one notification device. However, in other embodiments, snoozing may occur at multiple notification devices or at other devices where alerts may be triggered (e.g., a computer, a phone, etc.).

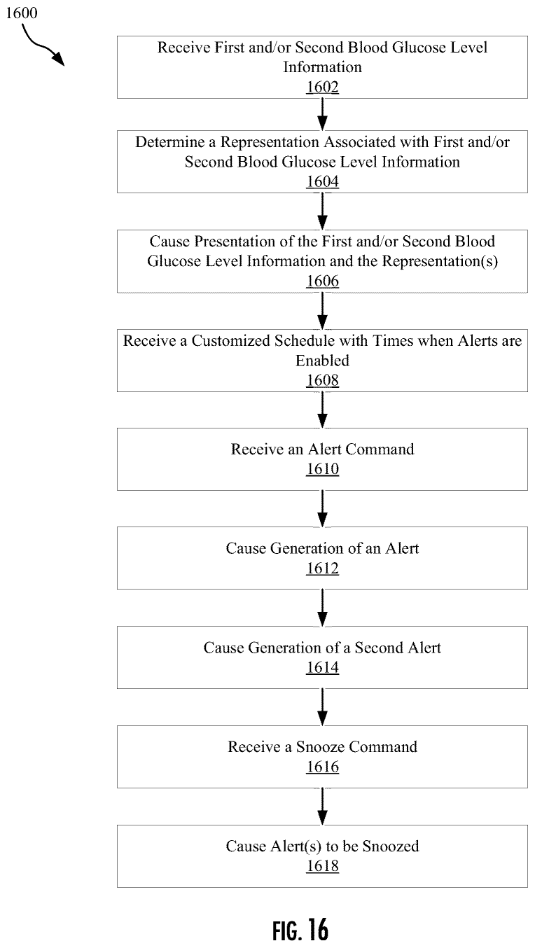

FIG. 16 illustrates an example flow chart with a method 1600 for presenting blood glucose level information for a tracked individual. At operation 1602, first blood glucose level information and/or second blood glucose level information may be received. The first blood glucose level information and the second blood glucose level information may each include at least one of a blood glucose level, a first derivative for the blood glucose level as a function of time, or a second derivative for the blood glucose level as a function of time. The first and second blood glucose level information may be received from a respective tracking device, with the tracking device being a continuous blood glucose monitor, an insulin pump, a smart insulin pen, or a similar device. Where first and second blood glucose level information are received, this information may be for two different tracked individuals (e.g., two children of a parent). In addition to the blood glucose level information, data from an insulin pump or a smart insulin pen may be received and displayed on devices.

In some embodiments, the first and/or second blood glucose level information may include data having specific blood glucose level information. However, in some embodiments, the first and/or second blood glucose level information may also include indication that the blood glucose level either falls inside or outside of an acceptable range, with these indications not including any specific blood glucose level. These indications may be received at a more regular frequency than the frequency at which data having specific blood glucose level information is received, allowing alerts to be generated more quickly if a tracked individual is outside of an acceptable range. The first and/or the second blood glucose level information may first be sent to a user device and/or a server before it is received at the notification device. The user device may be at least one of a computer, a cell phone, a smart phone, receivers, or a tablet.

At operation 1604, a representation associated with the first blood glucose level information and/or the second blood glucose level information may be determined. This determination may be made based on the first blood glucose level information and/or the second blood glucose level information received at operation 1602. The representation may include at least one of an emoji, a slang term, a color, a quote, or something similar. In some embodiments, the representation(s) may be customizable by a tracked individual, a user, or by another person so that customized representations may be presented for different blood glucose level ranges.

At operation 1606, presentation of the first and/or the second blood glucose level information and the associated representation(s) may be caused. Presentation of this material may be caused on the screen of a notification device, but presentation of this material may occur at another location in some embodiments.

At operation 1608, a customized schedule may be received with times when alerts are enabled. By doing so, alerts may only be generated during time periods when the user's customized schedule allows alerts, and alerts may be prevented during other time periods. However, in some embodiments, even when the user has disabled alerts during a particular time, alerts may still be generated if the tracked individual triggers the alert or if the blood glucose level information is extremely poor.

At operation 1610, an alert command may be received. This may be received by a tracked individual on a tracking device that the tracked individual is wearing, by the tracked individual or another user on a notification device, or by a tracked individual or another user on another device. The alert command may be generated upon an alert button being pressed by a user in some embodiments, but the alert command may be generated upon a voice command or some other method of actuation.

At operation 1612, generation of an alert may be caused, and the alert may be based on the first and/or the second blood glucose level information. The alert may comprise at least one of an audio alert, a visual alert, or a vibratory alert. The alert may be caused at a particular notification device, at another notification device (and not one where an alert command may have been triggered), or at another device.

At operation 1614, generation of a second alert may be caused. The alerts may be randomized to prevent brain habituation—for example, the second alert that is generated may be distinct from the first alert (e.g., in its sound, color, frequency, etc.). In some embodiments, if a user has not snoozed an alert after a certain duration of time, a second alert having a greater magnitude may be generated. The magnitude may be greater in terms of volume level, brightness, flashing frequency, prominence of a visual alert display, or in other ways. In some embodiments, if a user has not snoozed an alert after a certain duration of time, a second alert may be caused that sends a notification to another device, with the notification including a phone call, a text message, an email, and/or a notification alert.

At operation 1616, a snooze command may be received. In some embodiments, this snooze command may be received at a separate device from the notification devices described herein, such as from a remote computer or phone. However, in some embodiments, snooze commands may simply be received at a notification device.

At operation 1618, one or more alerts may be snoozed. In some embodiments, snoozing may occur only at one notification device. However, in other embodiments, snoozing may occur at multiple notification devices or at other devices where alerts may be triggered (e.g., a computer, a phone, etc.).

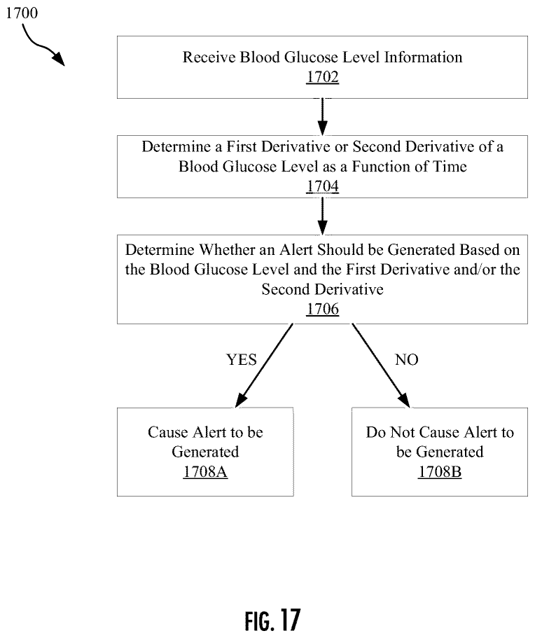

FIG. 17 illustrates an example flow chart with a method for presenting blood glucose level information for a tracked individual. At operation 1702, blood glucose level information may be received. The blood glucose level information may include a blood glucose level. The blood glucose level information may be received from a continuous blood glucose monitor, but other information or data may also be received at operation 1702, with this information or data being received from an insulin pump or a smart insulin pen.

At operation 1704, a determination of the first derivative and/or the second derivative of the blood glucose level as a function of time may be determined. Additionally, at operation 1706, a determination may be made as to whether an alert should be generated based on the blood glucose level and the first derivative and/or the second derivative. For example, a determination may be made that the alert should be generated when the blood glucose level is in a normal range but the first derivative and/or the second derivative for the blood glucose level indicate that the blood glucose level is about to exit the normal range. As another example, a determination may be made that the alert should not be generated where the blood glucose level falls outside of a normal range but a first derivative for the blood glucose level indicates that the blood glucose level is about to enter into the normal range. If it is determined that an alert should be generated at operation 1706, then the method 1700 may proceed to operation 1708A. If it is determined that no alert should be generated at operation 1706, then the method may proceed to operation 1708B. At operation 1708A, generation of an alert may be caused as described herein. At operation 1708B, generation of an alert may not be caused.

The methods described in FIGS. 15-17 are merely exemplary, and it should be understood that the methods may be adjusted in various ways. For example, the operations of the methods may be performed in different orders, and some of the operations may be performed simultaneously in some embodiments. Additionally, certain operations from the methods described in FIGS. 15-17 may be combined together (e.g., operations of method 1700 may be added into the method 1500 or the method 1600). Operations may also be omitted from the methods in some embodiments, and additional operations may be added in some embodiments as well.

### CONCLUSION

Many modifications and other embodiments set forth herein will come to mind to one skilled in the art to which these embodiments pertain having the benefit of the teachings presented in the foregoing descriptions and the associated drawings. Therefore, it is to be understood that the embodiments are not to be limited to the specific embodiments disclosed and that modifications and other embodiments are intended to be included within the scope of the invention. Moreover, although the foregoing descriptions and the associated drawings describe example embodiments in the context of certain example combinations of elements and/or functions, it should be appreciated that different combinations of elements and/or functions may be provided by alternative embodiments without departing from the scope of the invention. In this regard, for example, different combinations of elements and/or functions than those explicitly described above are also contemplated within the scope of the invention. Although specific terms are employed herein, they are used in a generic and descriptive sense only and not for purposes of limitation.

1. A notification device for presenting blood glucose level information for a tracked individual, wherein the notification device is configured to receive first blood glucose level information from a first tracking device associated with a first manufacturer, wherein the notification device is configured to receive second blood glucose level information from a second tracking device associated with a second manufacturer, wherein the first blood glucose level information and the second blood glucose level information each include at least one of a blood glucose level, a first derivative for the blood glucose level as a function of time, or a second derivative for the blood glucose level as a function of time, the notification device comprising:
a screen;
one or more processors; and
one or more memory devices containing computer readable code thereon that, when executed by the one or more processors, is configured to cause the one or more processors to:
receive at least one of the first blood glucose level information or the second blood glucose level information; and
cause presentation of at least one of the first blood glucose level information or the second blood glucose level information on the screen,
wherein the first tracking device and the second tracking device are each a continuous blood glucose monitor.

2. The notification device of claim 1, wherein the first blood glucose level information is associated with blood glucose levels for a first tracked individual, and wherein the second blood glucose level information is associated with blood glucose levels for a second tracked individual.

3. The notification device of claim 1, wherein the notification device is configured to generate alerts based on the first blood glucose level information, wherein the alerts include at least one of audio alerts, visual alerts, vibratory alerts, or notifications, and wherein the notifications are sent to another device and include at least one of a phone call, a text message, an email, or a notification alert.

4. The notification device of claim 3, wherein the notification device is configured to cause randomized alerts.

5. The notification device of claim 3, wherein the notification device is configured to enable customization of time periods when the alerts are enabled and when the alerts are disabled.

6. The notification device of claim 3, wherein the notification device is configured to enable snoozing of alerts.

7. The notification device of claim 6, wherein, when executed by the one or more processors, the computer readable code is configured to cause the one or more processors to:
receive a snooze command from another device; and
cause an alert to be snoozed based on the snooze command.

8. The notification device of claim 6, wherein, when executed by the one or more processors, the computer readable code is configured to cause the one or more processors to:
receive a snooze command; and
cause an alert at the notification device and a second alert at a second device to be snoozed based on the snooze command.

9. The notification device of claim 6, wherein, when executed by the one or more processors, the computer readable code is configured to cause the one or more processors to:
generate a first alert at a first time; and
generate a second alert at a second time after the first time,
wherein the second alert is greater in magnitude relative to the first alert when the notification device has not been snoozed between the first time and the second time.

10. The notification device of claim 1, wherein, when executed by the one or more processors, the computer readable code is configured to cause the one or more processors to:
receive an indication that the blood glucose level either falls inside or outside of an acceptable range, wherein the indication does not include a specific blood glucose level; and
cause an alert to be generated if the blood glucose level falls outside of the acceptable range.

11. The notification device of claim 10, wherein, when executed by the one or more processors, the computer readable code is configured to cause the one or more processors to:
receive a second indication including a specific blood glucose level,
wherein the indication is received at a greater frequency than the second indication.

12. The notification device of claim 1, wherein, when executed by the one or more processors, the computer readable code is configured to cause the one or more processors to:
receive data from an insulin pump; and
cause the data received from the insulin pump to be displayed.

13. The notification device of claim 1, wherein the notification device is configured to generate alerts indicating when limited data is available or when no data is available.

14. A method for presenting blood glucose level information for a tracked individual, the method comprising:
receiving at least one of first blood glucose level information or second blood glucose level information, wherein the first blood glucose level information is from a first tracking device associated with a first manufacturer, wherein the second blood glucose level information is from a second tracking device associated with a second manufacturer, and wherein the first blood glucose level information and the second blood glucose level information each include at least one of a blood glucose level, a first derivative for the blood glucose level as a function of time, or a second derivative for the blood glucose level as a function of time; and
causing presentation of at least one of the first blood glucose level information or the second blood glucose level information on a screen of a notification device,
wherein the first tracking device and the second tracking device are each a continuous blood glucose monitor.

15. The method of claim 14, wherein the first blood glucose level information is associated with blood glucose levels for a first tracked individual, and wherein the second blood glucose level information is associated with blood glucose levels for a second tracked individual.

16. The method of claim 14, further comprising:
causing generation of alerts based on the first blood glucose level information,
wherein the alerts include at least one of an audio alert, a visual alert, a vibratory alert, or a notification, and wherein the notification is sent to another device and includes at least one of a phone call, a text message, an email, or a notification alert.

17. The method of claim 16, wherein the alerts are randomized relative to each other.

18. The method of claim 16, further comprising:
receiving a customized schedule from a user with time periods when alerts are enabled and disabled,
wherein the generation of the alerts occurs in a time period when alerts are enabled in the customized schedule.

19. The method of claim 16, further comprising:
receiving a snooze command; and
causing the alerts to be snoozed based on the snooze command.

20. The method of claim 19, further comprising:
generating a first alert at a first time; and
generating a second alert at a second time after the first time when the notification device has not been snoozed between the first time and the second time,
wherein the second alert is greater in magnitude relative to the first alert.

21. The method of claim 16, further comprising:
receiving a snooze command from another device; and
causing an alert to be snoozed based on the snooze command.

22. The method of claim 16, further comprising:
receiving a snooze command; and
causing a first alert at the notification device and a second alert at a second device to be snoozed based on the snooze command.

23. The method of claim 14, further comprising:
receiving an indication that the blood glucose level either falls inside or outside of an acceptable range, wherein the indication does not include a specific blood glucose level; and
causing an alert to be generated if the blood glucose level falls outside of the acceptable range.

24. The method of claim 23, further comprising:
receiving a second indication including a specific blood glucose level,
wherein the indication is received at a greater frequency than the second indication.

25. The method of claim 14, further comprising:
receiving data from an insulin pump; and
causing the data received from the insulin pump to be displayed.

26. The method of claim 14, further comprising:
generating alerts indicating when limited data is available or when no data is available.

27-64. (canceled)
